(19) United States
(12) Patent Application Publication
(10) Pub. No.: US 2025/0295633 A1
(43) Pub. Date: Sep. 25, 2025

(54) USE OF ANTIANDROGENS TO TREAT SEPSIS

(71) Applicants: Ofer Andy GOREN, Prague (CZ); Rhode Island Hospital, Providence, RI (US)

(72) Inventors: Carlos G. Wambier, West Greenwich, RI (US); Gerard J. Nau, Sharon, MA (US); Ofer Andy Goren, Prague (CZ)

(21) Appl. No.: 19/081,887

(22) Filed: Mar. 17, 2025

Related U.S. Application Data
(60) Provisional application No. 63/568,983, filed on Mar. 22, 2024.

(51) Int. Cl. A61K 31/4178; A61P 11/00; A61P 31/14; C12Q 1/6883

(52) U.S. Cl. CPC A61K 31/4178; A61P 11/00; A61P 31/14; C12Q 1/6883; C12Q 2600/106; C12Q 2600/156; C12Q 2600/158

(57) ABSTRACT

The invention relates to a method for treating a condition and/or sepsis in a subject by administering a therapeutically effective amount of an anti-androgen small molecule. The method involves executing an analytical test to determine if the subject is an appropriate candidate for the treatment. If deemed suitable, the anti-androgen small molecule is administered, resulting in an improvement in the condition and/or sepsis compared to the subject without the treatment. The method also includes assessing the success of the treatment through various analytical tests performed at different time points after administration. The anti-androgen small molecule may include compounds such as sabizabulin, enzalutamide, or others, and is administered in a dose ranging from about 0.05 mg to about 2000 mg per day. The treatment is tailored based on the severity and stage of the condition and adjusted according to the subject's response.

### CROSS REFERENCE TO RELATED APPLICATIONS

This application claims the benefit of priority to U.S. Provisional Patent No. 63/568,983, filed 22 Mar. 2024, the entire disclosure of which is incorporated by reference as if fully set forth herein in its entirety.

### FIELD OF THE INVENTION

The present disclosure relates, for example, to methods for determining if a subject will respond positively to an anti-androgen therapy regime or not, specifically focusing on the treatment of sepsis using anti-androgen or other small molecules.

### STATEMENT REGARDING FEDERALLY SPONSORED RESEARCH OR DEVELOPMENT

Not applicable (N/A).

### BACKGROUND OF THE INVENTION

Previous approaches to treating sepsis and related conditions have primarily focused on the use of broad-spectrum antibiotics and supportive care to manage symptoms and prevent complications. These methods aim to control the infection and stabilize the patient, but they often do not address the underlying pathophysiological mechanisms that contribute to the progression of sepsis. Additionally, the overuse of antibiotics has led to increased antibiotic resistance, making it imperative to explore alternative therapeutic strategies.

In recent years, there has been growing interest in the role of hormonal pathways, particularly those involving androgens, in the modulation of immune responses and inflammation. Some studies have suggested that androgens may influence the severity of sepsis by affecting immune cell function and cytokine production. This has led to the exploration of anti-androgen therapies as a potential means to modulate these pathways and improve outcomes in septic patients. However, the application of anti-androgen therapies in sepsis treatment has been limited and not widely adopted in clinical practice.

In general, sepsis is a life-threatening condition that arises when the body's response to infection causes injury to its own tissues and organs. Despite advances in medical care, sepsis remains a major cause of morbidity and mortality worldwide. The complexity of sepsis, involving a dysregulated immune response to infection, makes it challenging to diagnose and treat effectively. Current treatment strategies primarily focus on controlling the infection with antibiotics and supporting the function of vital organs. However, these approaches do not address the underlying immune dysregulation that characterizes sepsis, highlighting the need for more targeted therapies.

Anti-androgen small molecules or small molecules with similar effects have traditionally been used in the treatment of conditions like prostate cancer, where they help to inhibit the effects of androgens, which can promote tumor growth. Recent research suggests that androgens may also play a role in the immune response, potentially influencing the progression of sepsis. This has led to interest in exploring the use of anti-androgen therapies as a novel approach to modulating the immune response in sepsis. What is particularly frustrating is that come subjects respond well to a specific anti-androgen therapy while other similar subjects do not respond well. Identifying which patients might benefit from such treatments remains a significant challenge, urgently necessitating the development of reliable methods disclosed herein to assess patient suitability for anti-androgen therapy in the context of sepsis or other condition.

### BRIEF SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

Sepsis is a life-threatening condition that arises when the body's response to infection causes injury to its own tissues and organs. Despite advances in medical care, sepsis remains a major cause of morbidity and mortality worldwide. The complexity of sepsis, involving a dysregulated immune response to infection, makes it challenging to diagnose and treat effectively. Current treatment strategies primarily focus on controlling the infection with antibiotics and supporting the function of vital organs. However, these approaches do not address the underlying immune dysregulation that characterizes sepsis, highlighting the need for more targeted therapies.

Anti-androgen small molecules have traditionally been used in the treatment of conditions like prostate cancer, where they help to inhibit the effects of androgens, which can promote tumor growth. Recent research suggests that androgens may also play a role in the immune response, potentially influencing the progression of sepsis. This has led to interest in exploring the use of anti-androgen therapies as a novel approach to modulating the immune response in sepsis. However, identifying which patients might benefit from such treatments remains a significant challenge, necessitating the development of reliable methods herein to assess patient suitability for anti-androgen therapy in the context of sepsis.

In the exploration of anti-androgen therapies for sepsis, it has been observed that patient responses can vary significantly. Some patients respond well to anti-androgen therapy, experiencing marked improvements in their condition. These patients often exhibit a reduction in the severity of sepsis symptoms (or signs and/or symptoms for other conditions) and a stabilization of vital organ functions. The positive response in these patients may be attributed to specific biological markers or genetic predispositions that make them more receptive to the effects of anti-androgen treatment. Understanding these factors is crucial for tailoring therapies to individual needs and maximizing treatment efficacy.

Conversely, there are patients who, despite having similar demographic characteristics, do not respond well to the same anti-androgen therapy. These patients may show little to no improvement, or in some cases, their condition may even worsen. The lack of response in these patients could be due to a variety of factors, including differences in the underlying pathophysiology of their sepsis, variations in androgen receptor expression, or other genetic and environmental influences that affect how their bodies process and respond to the therapy.

The variability in patient responses underscores the importance of personalized medicine in the treatment of sepsis. It highlights the need for comprehensive diagnostic tools that can accurately predict which patients are likely to benefit from anti-androgen therapy. Such tools would ideally assess a range of biomarkers, including genetic, proteomic, and metabolomic data, to provide a holistic view of the patient's condition and potential response to treatment.

The technology disclosed herein works for a broad range of conditions in addition to sepsis. A method for treating a condition and/or sepsis in a subject involves executing an analytical test to determine the subject's suitability for treatment with a therapeutically effective amount of an anti-androgen small molecule or a potential anti-androgen small molecule. Upon identifying the subject as an appropriate candidate, the method includes administering the therapeutically effective amount of the anti-androgen small molecule and/or the potential anti-androgen small molecule. This administration results in an improvement of the condition and/or sepsis in the subject compared to the absence of such treatment. The method provides a targeted approach to enhance therapeutic outcomes in subjects identified as suitable candidates through preliminary testing.

Dexamethasone is contemplated herein to have mild antiandrogen effects, by reducing the expression of adrenal corticosteroids, and keeping in mind possible combination therapies and the above discussion, as an additional brief summary or to provide discussion points for a brief summary, some example features of the technology disclosed herein can be briefly summarized by the following list of features, any of which can be inter-combined or discussed with any other feature, Figure, Drawing, detail, embodiment, aspect, or example disclosed herein:

Feature 1: A method for treating a condition and/or a sepsis in a subject in need thereof, the method comprising the steps of: (1) executing an analytical test on the subject, whereby the test will assess whether or not the subject is an appropriate candidate for a treatment by an administration of a therapeutically effective amount of an anti-androgen small molecule or a potential anti-androgen small molecule; (2) if the subject is an appropriate candidate after the test in step (1), administer the therapeutically effect amount of the anti-androgen small molecule and/or the potential anti-androgen small molecule to the subject; whereby the condition and/or the sepsis in the subject is improved compared to the same subject without the administering of the therapeutically effect amount of the anti-androgen small molecule and/or the potential anti-androgen small molecule to the subject.

Feature 2: The method of feature 1, further comprising the step of: (3) executing an analytical test to assess success of the administration of the anti-androgen small molecule or a potential anti-androgen small molecule, wherein the analytical test comprises an RNA sequencing test, a radiological test, a dexamethasone androgen suppression test, a COVID-19 test, a culture test, a nasopharyngeal viral clearance test, a protein expression test, a biomarker test, a clinical symptom assessment, or a combination thereof; and/or determining a clinical score based on a physical examination for evaluation of signs of hyperandrogenic features such as evaluating body/face/scalp/pubic hair, oily skin, enlarged external genital (prostate/penis/clitoris); or a combination of clinical and laboratory criteria. According to some aspects, the laboratory and/or clinical scores can be used for screening an individual in step 100, 102, and/or 104 of FIG. 1.

Feature 3: The method of feature 1, wherein the small molecule comprises sabizabulin or a pharmaceutically acceptable hydrate, solvate, and/or salt form of sabizabulin.

Feature 4: The method of feature 1, wherein the anti-androgen small molecule or the potential anti-androgen small molecule is administered in a therapeutically effective amount that is determined based on the severity and stage of the condition and/or sepsis in the subject.

Feature 5: The method of feature 1, wherein the improvement in the condition and/or sepsis is compared to the same subject without the administering of the therapeutically effective amount of the anti-androgen small molecule or the potential anti-androgen small molecule, and wherein the improvement is measured by one or more clinical parameters or biomarkers.

Feature 6: The method of feature 1, wherein the anti-androgen small molecule or the potential anti-androgen small molecule is at least one of an anti-androgen small molecule or a potential anti-androgen small molecule that acts on the androgen receptor pathway.

Feature 7: The method of feature 1, wherein the analytical test substantially assesses the appropriateness of the subject for the treatment by measuring one or more biomarkers, clinical symptoms, or risk factors associated with the condition and/or sepsis.

Feature 8: The method of feature 1, wherein the administering is substantially of the therapeutically effective amount of the anti-androgen small molecule or the potential anti-androgen small molecule, and wherein the therapeutically effective amount is sufficient to improve the condition and/or sepsis in the subject.

Feature 9: The method of feature 1, wherein the condition comprises COVID-19 infection, a traumatic lung injury, a skin condition, or any sign, symptom or condition that mimics a sepsis infection.

Feature 10: The method of feature 1, wherein the condition and/or sepsis is one or more of a bacterial infection, viral infection, fungal infection, parasitic infection, inflammatory condition, or sepsis.

Feature 11: The method of feature 1, wherein the subject is a human patient diagnosed with or at risk of developing the condition and/or sepsis.

Feature 12: The method of feature 1, wherein the analytical test is performed prior to the administering and is used to select subjects who are likely to respond to the treatment.

Feature 13: The method of feature 1, wherein the administering is performed after the assessing and only in subjects who are determined to be appropriate candidates for the treatment based on the results of the analytical test.

Feature 14: The method of feature 1, wherein the improvement is assessed after the administering at one or more time points to monitor the effectiveness of the treatment.

Feature 15: The method of feature 1, wherein the anti-androgen small molecule or the potential anti-androgen small molecule is administered orally, intravenously, intramuscularly, or subcutaneously using a suitable pharmaceutical formulation.

Feature 16: The method of feature 1, wherein the anti-androgen small molecule or the potential anti-androgen small molecule is administered in a single dose or in multiple doses according to a therapeutic regimen that is tailored to the individual subject.

Feature 17: The method of feature 1, wherein the therapeutically effective amount is determined based on the severity of the condition and/or sepsis in the subject, as well as other clinical factors such as age, weight, and overall health of the subject.

Feature 18: The method of feature 1, wherein the anti-androgen small molecule or the potential anti-androgen small molecule is selected based on the results of the analytical test, and wherein different anti-androgen small molecules or potential anti-androgen small molecules may be selected for different subjects depending on their individual test results.

Feature 19: The method of feature 1, wherein the analytical test assesses whether or not the subject is an appropriate candidate for the treatment by evaluating one or more genetic, biochemical, or clinical parameters that are predictive of the subject's response to the treatment.

Feature 20: The method of feature 1, wherein the therapeutically effective amount is administered to the subject assessed as an appropriate candidate for the treatment, and wherein the subject is monitored for adverse reactions or side effects during the course of the treatment.

In some embodiments, a method for treating a condition and/or a sepsis in a subject in need thereof is provided, the method comprising steps of: (1) executing an analytical test on the subject, whereby the test will assess whether or not the subject is an appropriate candidate for a treatment by an administration of a therapeutically effective amount of an anti-androgen small molecule or a potential anti-androgen small molecule; (2) if the subject is an appropriate candidate after the test in step (1), administer the therapeutically effect amount of the anti-androgen small molecule and/or the potential anti-androgen small molecule to the subject; and whereby the condition and/or the sepsis in the subject is improved compared to a ceteris paribus subject without the administering of the therapeutically effect amount of the anti-androgen small molecule and/or the potential anti-androgen small molecule to the subject.

According to some aspects, the method is further comprising the step of: (3) executing an analytical test to assess success of the administration of the anti-androgen small molecule or a potential anti-androgen small molecule, wherein the analytical test comprises an RNA sequencing test, a radiological test, a dexamethasone androgen suppression test, a COVID-19 test, a culture test, a nasopharyngeal viral clearance test, a protein expression test, a biomarker test, a clinical symptom assessment, or a combination thereof; and/or determining a clinical score based on a physical examination for evaluation of signs of hyperandrogenic features such as evaluating body/face/scalp/pubic hair, oily skin, enlarged external genital (prostate/penis/clitoris); or a combination of clinical and laboratory criteria. According to some aspects, the laboratory and/or clinical scores can be used for screening an individual in step 100, 102, and/or 104 of FIG. 1.

In some embodiments, the methods is wherein the improvement in the condition and/or sepsis is compared to the ceteris paribus subject without the administering of the therapeutically effective amount of the anti-androgen small molecule or the potential anti-androgen small molecule, and wherein the improvement is measured by one or more clinical parameters or biomarkers.

Research is ongoing herein to identify any additional biomarkers and develop predictive models that can guide treatment decisions. By integrating data from clinical trials, patient histories, and advanced computational analyses, researchers aim to create algorithms that can stratify patients based on their likelihood of responding to anti-androgen therapy. This approach not only promises to improve outcomes for patients with sepsis but also to reduce the risk of adverse effects associated with inappropriate treatment.

While anti-androgen therapy holds promise as a treatment for sepsis, its success is contingent upon the ability to identify and select appropriate candidates for the therapy. The development of reliable diagnostic methods to assess patient suitability is essential for the effective implementation of this treatment strategy. As research progresses, it is hoped that these efforts will lead to more personalized and effective interventions for sepsis, ultimately improving patient outcomes and reducing the global burden of this complex condition. Analytical tests to identify appropriate candidates for specific therapies are employed herein. These tests aim to tailor treatments based on individual patient characteristics, thereby improving efficacy and reducing adverse effects. In the context of sepsis, however, the integration of such diagnostic tools to guide the use of anti-androgen therapies remains in the future of this technology.

Any of the features, methods and/or details herein can be provided in a kit. While the summary examples disclosed above provide some introduction to embodiments of the invention, other implementations are also contemplated, described, and recited herein. These and other features and advantages will be apparent from a reading of the following detailed description, the example claims, and a review of the associated drawings. It is to be understood that both the foregoing general description and the following detailed description are explanatory only and are not restrictive of aspects as claimed.

### BRIEF DESCRIPTION OF THE DRAWINGS

For the purpose of illustration, certain discernable embodiments of the present invention are shown in the drawings described below. It should be understood, however, that the invention is not limited to the precise arrangements, data, dimensions, and illustrations shown. In the drawings:

FIG. 1 illustrates a flowchart for assessing treatment candidacy and administering anti-androgen therapy or a potential anti-androgen therapy.

It should be understood that while illustrations can sometimes be used in the example FIGURE above to describe different embodiments and different aspects of the technology, any aspect from any figure can be inter-combined with an aspect from any other figures or text. Any example disclosed herein can be inter-combined with any other. All trademarks, images, likenesses, words, and depictions that could be construed in the drawings and the disclosure are plainly in fair use and are provided solely for the purposes of illustration of the invention in view of an urgent need to prevent injuries and to treat subjects as further discussed in more detail below.

### DETAILED DESCRIPTION OF THE INVENTION

The subject innovation is now described, in some examples with reference to the drawings, wherein examples can used to refer to the aspects of the breadth of concepts of the invention. In the following description, for purposes of explanation, specific details are set forth in order to provide a thorough understanding of the present invention. It may be evident, however, that the present invention may be practiced without these specific details. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention can be determined by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, a “range” may be provided. A statement may include “in the range from about A to about B”. All points from A to B are subsumed by the range, and all those points can define preferred ranges. Within said range, any range subsumed therein means any range that is within the stated range. Endpoints within the range can define a new range. For example, the following are all subsumed within the range of about 10 to about 50. 10 to 20; 15 to 35; 23 to 40; or 50 to 31; or any other range or set of ranges within the stated range. As such, within the range any set of endpoints subsumed therein can be used as an exemplary range.

As used herein, the term “comprising” means that other elements can also be present in addition to the defined elements presented. The use of “comprising” indicates inclusion rather than limitation. Any method described herein can be claimed and/or described as a composition and vice versa.

The term “consisting of” as it is known in the practice refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention. In specific examples, “consisting essentially of” can be explained herein for each example or can be defined broadly, for example, by stating that an administration to a subject (in a method herein) does not include any other active pharmaceutical ingredient or therapeutic agent in addition to the one specified. In another example, the term “consisting essentially of” can be utilized to indicate a nanocarrier and a therapeutic agent with no other ingredients that are listed in a claim and yet including any other ingredients that are not specifically listed. That is, due to the life-saving advantages the technology herein presents, the term “consisting essentially of” can be utilized to exclude any additional method step(s) and/or ingredient(s) that can be discussed (or listed); for example, a claim can be directed to 1. A method for treating a condition and/or a sepsis in a subject in need thereof, the method comprising the steps of: (1) executing an analytical test on the subject, whereby the test will assess whether or not the subject is an appropriate candidate for a treatment by an administration of a therapeutically effective amount of an anti-androgen small molecule or a potential anti-androgen small molecule; (2) if the subject is an appropriate candidate after the test in step (1), administer the therapeutically effect amount of the anti-androgen small molecule and/or the potential anti-androgen small molecule to the subject; whereby the condition and/or the sepsis in the subject is improved compared to the same subject without the administering of the therapeutically effect amount of the anti-androgen small molecule and/or the potential anti-androgen small molecule to the subject; and this same claim can include a “proviso”, list or condition within that any ingredient (i.e., that can be discussed or listed) or method step is not included in the method and/or composition while retaining the condition that any other method step and/or ingredient that is not listed can be included with the method. This stipulation is intended to save lives by application of this technology. Thus, the technology is not to be subjected to limitations that could be normally applied to strict specific definitions and/or examples required for “consisting essentially of” (e.g., the need to define specific examples in this specification) so that increased lives are saved via applications of the Invention's claims.

The term “statistically significant” or “significantly” refers to statistical significance and generally means a two-standard deviation (2SD) or greater difference. The term “feature” and the term “detail” can be interchanged with a “claim”. Any list of features, details, examples, embodiments, and/or aspects herein can be placed into a “claim”.

As used herein, the term “subject” refers to a mammal, bird, or the like, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), racing mammals, laboratory animals (e.g., mice, rats, rabbits, etc.), but are not so limited. Subjects include human subjects. The human subject may be a pediatric, adult, or a geriatric subject. The human subject may be of either sex. In another example, the term “subject” can refer to a connective tissue culture, and the methods disclosed herein, while claimed towards subjects, contemplate use in the laboratory in synthetic tissue(s). As used herein, a female cell can refer to a cell with 2X chromosomes; a male cell can refer to a cell with 1X and 1Y chromosome.

As used herein, the terms “effective amount” and “therapeutically effective amount” include an amount sufficient to modulate a treatment or prevent or ameliorate a manifestation of disease or medical condition, such as a connective tissue condition or a risk of a connective tissue injury. Such a condition (or risk) may not be readily discernable and may take years, statistical analysis, and/or machine learning to determine a prevention, treatment, or amelioration. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the condition and on the characteristics of the individual, such as general health, age, sex, body weight and tolerance to drugs. It will also depend on the degree, severity and type of condition. The skilled artisan will be able to determine appropriate dosages depending on these and other factors. The compositions can also be administered in combination with one or more additional therapeutic compounds.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, manage, modulate, slow down or stop the progression or severity of a symptom or condition. The term “treating” includes reducing or alleviating at least one adverse effect (undesirable characteristic) or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a condition or decay, delay or slowing of a progression and/or risk of injury, and an increased lifespan/enjoyment as compared to that expected in the absence of treatment.

As used herein, the term “long-term” administration means that the therapeutic agent or drug is administered for a period of at least 12 weeks. The therapeutic agent or drug may refer to a formulation, composition, or agent. The formulation can be changed to a fresh formulation during administration. This includes that the therapeutic agent or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by any technique known in the art that is subsequently applied to a subject, topical application, absorption, injection, ingestion, transfusion, implantation or transplantation. In an example embodiment, compositions are applied as a tablet or drug in capsule. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subdermal, subcuticular, intraarticular, subcapsular, subarachnoid, intraspinal and intrasternal injection and infusion. It is known in the art that therapeutic agents can be rapidly deployed through the skin and directly into joint/ligaments by use of DMSO (dimethyl sulfoxide) as a carrier solvent applied (with the therapeutic agent) to the skin near to or surrounding a joint. While DMSO is rarely used anymore for these purposes because of its nature as a universal solvent and its tendency to carry any residual chemicals present on the skin into the bloodstream (along with the intended agent), the technology contemplates such uses. In one contemplated embodiment, the compositions contemplated herein are administered to a subject by direct injection into a tissue, lymph node, or site of treatment. In another example, administration is provided in the form of a natural product, vitamin, supplement, food, aerosol, inhalation, vapor, or drink. Formulations disclosed herein can be ready made or require mixing just before administration.

Any of the methods disclosed herein can be carried out in part or completely by including a dietary change, a food, natural product, precursor, or prodrug of a therapeutic agent. As used herein, a precursor or a prodrug is intended to encompass compounds or therapeutic agents which, under physiologic conditions, are converted into the therapeutically active agents of the present invention (e.g., a compound for any of the present claims or features). A common method for making a prodrug is to include one or more selected moieties which are hydrolyzed under physiologic conditions to reveal the desired molecule. In other embodiments, the prodrug is converted by an enzymatic activity of the host subject. For example, esters or carbonates (e.g., esters or carbonates of alcohols or of carboxylic acids) are preferred prodrugs of the present invention. In certain embodiments, some or all of the small-molecule chemical structures selected from this disclosure can be replaced with the corresponding suitable prodrug, for example, wherein a hydroxyl in the parent compound is presented as an ester or a carbonate or carboxylic acid present in the parent compound is presented as an ester. A common method of making a precursor/prodrug that can be used herein is to use a carrier/nanocarrier (e.g., mesoporous silica particles). The precursor/prodrug can be released from a carrier to form the active therapeutic agent. A precursor or prodrug can be metabolized to the active parent compound (therapeutic agent) in vivo (e.g., the ester is hydrolyzed to the corresponding hydroxyl, or carboxylic acid). No argument can be made that the term “prodrug” is not enabled herein based on an assertion that actual prodrugs were not made and tested.

The terms: “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms: “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, the term: “small molecule” refers to a molecule that has a molecular weight <1000. As used herein, the term: “large molecule” refers to a molecule that has a molecular weight >1000, and the term includes biologics such as the examples of oligonucleotides, peptides, antibodies, linkers, oligosaccharides, polymers, DNA chains, and RNA chains. The term: “therapeutic agent” may refer to small molecule, element, large molecule, biologic, formulation, composition, agent, or a combination thereof.

### Pharmaceutical Compositions

The compositions and methods of the present invention may be utilized to prevent a need for other treatment, to provide benefit when other treatment(s) fail, or to treat an individual in need thereof. In some embodiments, the individual is suspected of needing treatment. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. A compound can represent a combination therapy herein. Pharmaceutically acceptable carriers are well known in the art and include, for example, aqueous solutions such as water or physiologically buffered saline or other solvents or vehicles such as glycols, glycerol, oils such as olive oil, or injectable organic esters. In some embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible compositions employed in pharmaceutical formulations.

A pharmaceutical composition (preparation) can be administered to a subject by any of a number of routes of administration including, for example, orally, for example, drenches as in aqueous or non-aqueous solutions or suspensions, tablets, capsules including sprinkle capsules and gelatin capsules, boluses, powders, granules, pastes for application to the tongue; absorption through the oral mucosa (e.g., sublingually); subcutaneously; transdermal administration (for example as a patch applied to the skin); and topically (for example, as a cream, ointment or spray applied to the skin). The compound may also be formulated for inhalation. Inhalation can include inhalation of a liquid (droplets or aerosol). Inhalation can include a micronized powder adhered to carrier particles or can be without carrier particles. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound (or combination therapy) of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules, including sprinkle capsules and gelatin capsules), tablets, pills, dragées, powders, granules and the like, the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragées, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymers and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art. See, e.g., Isselbacher, et al., (1996).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The subject or patient receiving this treatment is any animal in need, including primates, in particular humans; and other mammals such as equines bovine, porcine, sheep, feline, and canine; bird such as poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I-tartaric acid, thiocyanic acid, p-toluenesulfonic acid, trifluoroacetic acid, and undecylenic acid salts. In some embodiments, an N—H in elraglusib can become charged and a corresponding salt formed at or near the charge.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent. The invention contemplates polymorphs, cocrystals, and amorphous forms of all substances discussed herein. As discussed above, solvates and/or hydrates can be formed by, for example, a slow evaporation whereby water and/or solvent remain hydrogen bonded with OH groups in the molecule. The formation of a solvate/hydrate can be quickly confirmed after the evaporation by using attenuated total reflectance Fourier transform infra-red spectroscopy (ATR-IR) wherein the solid solvate/hydrate is directly placed on the instrument and the subsequent IR spectrum is compared to the IR spectrum of the solid non-solvate, non-hydrate. Any of the constructs herein can be utilized with solvation, targeting moieties, hydration, and/or with lipid formulations. No reasonable argument can be made that a solvate and/or hydrate is not enabled herein because specific chemical compositions were not made. It is known in the art that an ATR-IR can be utilized to rapidly make a solvate and/or hydrate. Similarly, no reasonable argument can be made that a salt is not enabled by this disclosure.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

As discussed above, unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy; The Encyclopedia of Molecular Cell Biology and Molecular Medicine; Molecular Biology and Biotechnology: a Comprehensive Desk Reference; Immunology; Janeway's Immunobiology; Lewin's Genes XI; Molecular Cloning: A Laboratory Manual.; Basic Methods in Molecular Biology; Laboratory Methods in Enzymology; Current Protocols in Molecular Biology (CPMB); Current Protocols in Protein Science (CPPS); and Current Protocols in Immunology (CPI).

In the embodiments discussed and in any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention or are used as would be understood by an ordinary person.

### Use of Antiandrogens to Treat Sepsis:

Disclosed now and in the inter-combinable Figures/Features herewith are new methods. In some examples, it is very difficult to predict how a patient will respond to an anti-androgen therapy or a potential anti-androgen therapy. The technology herein, according to some aspects, can provide a method for treating a condition and/or a sepsis in a subject in need thereof, the method comprising the steps of: (1) executing an analytical test on the subject, whereby the test will assess whether or not the subject is an appropriate candidate for a treatment by an administration of a therapeutically effective amount of an anti-androgen small molecule or a potential anti-androgen small molecule; (2) if the subject is an appropriate candidate, administer the therapeutically effect amount of the anti-androgen small molecule and/or the potential anti-androgen small molecule to the subject; whereby the condition and/or the sepsis in the subject is improved compared to the same subject without the administering of the therapeutically effect amount of the anti-androgen small molecule and/or the potential anti-androgen small molecule to the subject.

Sepsis is known as a leading cause of death globally. According to the United States Centers for Disease Control, at least 1.7 million adults in America develop sepsis annually, nearly 270,000 people. One out of every three people who die in a hospital die of sepsis. Animal studies and human observational studies showed higher rates of sepsis and worse outcomes in males. Males also have worse outcomes in other conditions that mimic sepsis, such as hemorrhagic shock and trauma. More recently, similar gender disparities were seen with COVID-19, with men faring worse than women.

The mainstay of sepsis management is treating the underlying infection. The Surviving Sepsis Campaign evaluated several interventions without identifying treatments with a meaningful impact on survival. Data from Brazil shows a dramatic impact of proxalutamide in reducing mortality in COVID-19 patients.

Similar gender disparities were seen with COVID-19 with men faring worse than women. The mainstay of sepsis management is treating the underlying infection. There remains a need in the biomedical art for better medical management of sepsis and COVID-19.

Importantly, any of the methods and or treatments herein can be inter-combined with measuring serum aliquots or tissue or fluid samples (e.g., saliva, urine, for proteins from genes promoted by the Androgen receptor: TMPRSS2, PSA (KLK3). Also, many other genes are directly or indirectly related to AR activation, cytokines: CXCL8, IL6; skin: keratin 5.

In some embodiments, the technology disclosed herein provides a therapeutic method for administering sabizabulin to treat sepsis and conditions that mimic sepsis, such as COVID-19 infections. In a first step, the method comprises an analytical test to assess whether the subject or patient is an appropriate candidate for treatment by the administration of sabizabulin or a therapeutically similar antiandrogen. In another step, the method comprises the administration of sabizabulin or a therapeutically similar antiandrogen to the appropriate candidate for treatment. Sabizabulin is an antiandrogen medication that blocks the translocation of the androgen receptor (AR) to the nucleus. Antiandrogens are candidates against the severe acute respiratory syndrome coronavirus 2 (SARS-CoV-2) disease (COVID-19) due to host cell entry inhibition by the suppression of TMPRSS2. Sabizabulin is under investigation for treating metastatic prostate cancer. In yet another step, the method may optionally comprise an analytical test to assess the progress or success of the administration of sabizabulin or therapeutically similar antiandrogens.

Many sepsis patients need 02. The main criteria for the use of antiandrogens is the patient use of 02 when they are in inflammatory state.

In some embodiments, this disclosure provides a method for treating a condition, including but not limited to sepsis, in a subject who may require such treatment. The method includes conducting an analytical test on the subject to determine their suitability for treatment with a therapeutically effective amount of an anti-androgen small molecule or a potential anti-androgen small molecule. If the subject is deemed suitable, the therapeutically effective amount of the anti-androgen small molecule and/or the potential anti-androgen small molecule may be administered to the subject. This administration can improve the condition, including sepsis, in the subject compared to the same subject without such administration. Various anti-androgen small molecules may be used, including sabizabulin, enzalutamide, apalutamide, darolutamide, relugolix, or their pharmaceutically acceptable forms.

The analytical test may include a wide range of assessments to determine the subject's suitability for treatment. In addition to RNA sequencing, the test may involve radiological tests, dexamethasone androgen suppression tests, COVID-19 tests, culture tests, nasopharyngeal viral clearance tests, protein expression tests, biomarker tests, clinical symptom assessments, or a combination thereof. The method can be customized to the individual subject, with the therapeutically effective amount adjusted based on the subject's response to treatment.

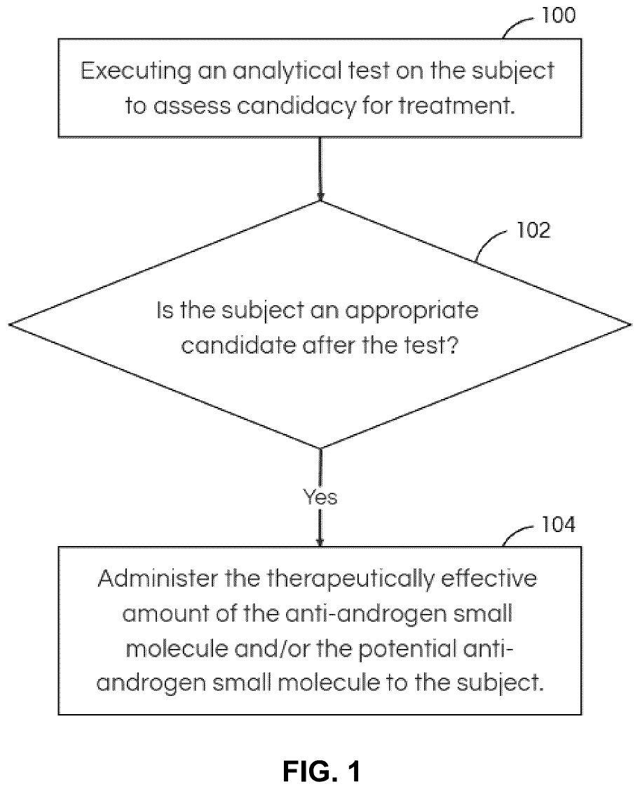

FIG. 1 is a flowchart illustrating a method for treating a condition, including sepsis, in a subject, according to an embodiment. Initially, an analytical test may be conducted on the subject. In some embodiments, this test includes an analytical or lab testing and/or determining a clinical score based on a physical examination for evaluation of signs of hyperandrogenic features such as evaluating body/face/scalp/pubic hair, oily skin, enlarged external genital (prostate/penis/clitoris); or a combination of clinical and laboratory criteria. According to some aspects, the laboratory and/or clinical scores can be used for screening an individual in step 100, 102, and/or 104 of FIG. 1. This test assesses whether the subject is suitable for a treatment involving the administration of a therapeutically effective amount of an anti-androgen small molecule or a potential anti-androgen small molecule. The test may evaluate the appropriateness of the subject for the treatment by measuring one or more biomarkers, clinical symptoms, or risk factors. It is performed prior to administration and is used to select subjects likely to respond to the treatment. The anti-androgen small molecule or the potential anti-androgen small molecule may be chosen based on the test results. The test evaluates one or more genetic, biochemical, or clinical parameters to ensure the subject is a suitable candidate for the treatment, potentially improving the condition, including sepsis, in the subject.

In the subsequent step, the process involves administering a therapeutically effective amount of an anti-androgen small molecule or a potential anti-androgen small molecule to a subject identified as suitable following an analytical test. This administration aims to treat a condition, including sepsis, in the subject. The anti-androgen small molecule or potential anti-androgen small molecule may include compounds such as sabizabulin, enzalutamide, apalutamide, darolutamide, or relugolix, among others. The administration is tailored based on the severity and stage of the condition, including sepsis, and the therapeutically effective amount may be adjusted during the course of treatment based on the subject's response. Improvement in the condition, including sepsis, may be measured by clinical parameters or biomarkers, which may include body temperature, heart rate, respiratory rate, blood pressure, oxygen saturation, and other relevant indicators. The anti-androgen small molecule or potential anti-androgen small molecule may act on the androgen receptor pathway, potentially serving as an androgen receptor antagonist, androgen synthesis inhibitor, or selective androgen receptor modulator. Administration may be performed through various routes, such as orally, sublingually, buccally, intranasally, rectally, topically, transdermally, subcutaneously, intramuscularly, intravenously, or by inhalation, using a suitable pharmaceutical formulation. The treatment regimen may involve a single dose or multiple doses, depending on the individual subject's needs. The subject may be a human patient diagnosed with or at risk of developing the condition, including sepsis, and the treatment may be initiated after assessing the subject's suitability for the treatment. Improvement in the condition, including sepsis, may be assessed at one or more time points to monitor the effectiveness of the treatment.

In addition to sepsis, the method may be applicable to a wide variety of other conditions. These conditions may include, but are not limited to, bacterial infections, viral infections, fungal infections, parasitic infections, inflammatory conditions, COVID-19 infection, traumatic lung injury, skin conditions, pneumonia, acute respiratory distress syndrome, influenza, meningitis, encephalitis, pancreatitis, appendicitis, cholecystitis, pyelonephritis, cellulitis, necrotizing fasciitis, toxic shock syndrome, septic arthritis, osteomyelitis, endocarditis, and bacteremia. The condition may be caused by a bacterial, viral, fungal, or parasitic pathogen. The method is particularly relevant for conditions associated with an elevated level of androgen receptor activity in the subject.

The analytical test may be performed prior to the administering and is used to select subjects who are likely to respond to the treatment. The test may be repeated during the course of treatment to monitor the subject's response. The administering is performed after the assessing and only in subjects who are determined to be appropriate candidates for the treatment based on the results of the analytical test. The administering may be initiated within 48 hours of the onset of symptoms of the condition, including sepsis. The improvement is assessed after the administering at one or more time points to monitor the effectiveness of the treatment. The improvement is assessed using one or more clinical parameters, biomarkers, or imaging modalities.

Referring again to the flowchart in FIG. 1, initially, an analytical test may be conducted on the subject (Step 100). In some embodiments, the analytical test can be performed at step 100, step 102, and/or at step 104; and/or determining a clinical score based on a physical examination for evaluation of signs of hyperandrogenic features such as evaluating body/face/scalp/pubic hair, oily skin, enlarged external genital (prostate/penis/clitoris); or a combination of clinical and laboratory criteria. According to some aspects, the laboratory and/or clinical scores can be used for screening an individual in step 100, 102, and/or 104 of FIG. 1. This test assesses whether the subject is suitable for a treatment involving the administration of a therapeutically effective amount of an anti-androgen small molecule or a potential anti-androgen small molecule. The test may evaluate the appropriateness of the subject for the treatment by measuring one or more biomarkers, clinical symptoms, or risk factors. It is performed prior to administration and is used to select subjects likely to respond to the treatment. The anti-androgen small molecule or the potential anti-androgen small molecule may be chosen based on the test results. The test evaluates one or more genetic, biochemical, or clinical parameters to ensure the subject is a suitable candidate for the treatment, potentially improving the condition and/or sepsis in the subject.

In the subsequent step (Step 102, FIG. 1), the process involves administering a therapeutically effective amount of an anti-androgen small molecule or a potential anti-androgen small molecule to a subject identified as suitable following an analytical test. This administration aims to treat a condition and/or sepsis in the subject. The anti-androgen small molecule or potential anti-androgen small molecule may include compounds such as sabizabulin, enzalutamide, apalutamide, darolutamide, or relugolix, among others. The administration is tailored based on the severity and stage of the condition and/or sepsis, and the therapeutically effective amount may be adjusted during the course of treatment based on the subject's response. Improvement in the condition and/or sepsis may be measured by clinical parameters or biomarkers, which may include body temperature, heart rate, respiratory rate, blood pressure, oxygen saturation, and other relevant indicators. The anti-androgen small molecule or potential anti-androgen small molecule may act on the androgen receptor pathway, potentially serving as an androgen receptor antagonist, androgen synthesis inhibitor, or selective androgen receptor modulator. Administration may be performed through various routes, such as orally, sublingually, buccally, intranasally, rectally, topically, transdermally, subcutaneously, intramuscularly, intravenously, or by inhalation, using a suitable pharmaceutical formulation. The treatment regimen may involve a single dose or multiple doses, depending on the individual subject's needs. The subject may be a human patient diagnosed with or at risk of developing the condition and/or sepsis, and the treatment may be initiated after assessing the subject's suitability for the treatment. Improvement in the condition and/or sepsis may be assessed at one or more time points to monitor the effectiveness of the treatment.

In Step 104 (FIG. 1), the method includes monitoring the subject's response to the treatment. This involves conducting follow-up assessments to evaluate the effectiveness of the administered anti-androgen small molecule or potential anti-androgen small molecule. The monitoring process may include repeated analytical tests to assess the success of the treatment and to make any necessary adjustments to the therapeutically effective amount. The subject's clinical parameters and biomarkers are evaluated to determine the progress and effectiveness of the treatment, ensuring that the condition and/or sepsis is being effectively managed. The anti-androgen small molecule or the potential anti-androgen small molecule is administered in a single dose or in multiple doses according to a therapeutic regimen that is tailored to the individual subject.

The therapeutic regimen comprises administering the anti-androgen small molecule or potential anti-androgen small molecule once, twice, or three times daily for a period of at least one week. The therapeutically effective amount is determined based on the severity of the condition, including sepsis, in the subject, as well as other clinical factors such as age, weight, and overall health of the subject. The therapeutically effective amount is adjusted based on the subject's response to the treatment as assessed by one or more clinical parameters or biomarkers.

The anti-androgen small molecule or the potential anti-androgen small molecule is selected based on the results of the analytical test. Different anti-androgen small molecules or potential anti-androgen small molecules may be selected for different subjects depending on their individual test results. The anti-androgen small molecule or potential anti-androgen small molecule may be administered alone or in combination with one or more additional therapeutic agents. The one or more additional therapeutic agents are selected from the group consisting of antibiotics, antiviral agents, antifungal agents, anti-inflammatory agents, immunomodulatory agents, and supportive care agents.

The analytical test assesses whether or not the subject is an appropriate candidate for the treatment by evaluating one or more genetic, biochemical, or clinical parameters that are predictive of the subject's response to the treatment. The one or more genetic, biochemical, or clinical parameters are selected from the group consisting of androgen receptor expression levels, androgen receptor mutation status, circulating androgen levels, inflammatory biomarkers, and clinical severity scores. The therapeutically effective amount is administered to the subject assessed as an appropriate candidate for the treatment. The subject is monitored for adverse reactions or side effects during the course of the treatment. The treatment is modified or discontinued if the subject experiences an adverse reaction or side effect that outweighs the potential benefit of the treatment.

The dexamethasone androgen suppression test is a diagnostic procedure used to evaluate the source of androgen excess in individuals with conditions such as polycystic ovary syndrome (PCOS). This test involves the administration of dexamethasone, a synthetic glucocorticoid, to suppress adrenal androgen production. The test is based on the principle that dexamethasone suppresses the pituitary release of adrenocorticotropic hormone (ACTH), which in turn reduces adrenal androgen secretion. By measuring androgen levels before and after dexamethasone administration, clinicians can determine whether the source of androgen excess is adrenal or ovarian.

In the context of functionally atypical polycystic ovary syndrome, the dexamethasone androgen suppression test can be combined with a low-dose ACTH test to further elucidate the source of androgen excess. The short dexamethasone androgen-suppression test involves administering dexamethasone over a short period, typically 2-3 days, and measuring serum androgen levels, such as testosterone and dehydroepiandrosterone sulfate (DHEAS), before and after the test. A significant reduction in androgen levels following dexamethasone administration suggests an adrenal source of androgen excess, while a lack of suppression indicates an ovarian source.

The low-dose ACTH test complements the dexamethasone suppression test by assessing adrenal responsiveness to ACTH stimulation. In this test, a low dose of ACTH is administered, and serum cortisol and androgen levels are measured at baseline and at intervals after ACTH administration. An exaggerated response in androgen levels to ACTH stimulation may indicate adrenal hyperactivity or hyperplasia.

Together or alone, these tests can be used in combination with the technology herein and provide valuable information about the source of androgen excess in individuals with functionally atypical PCOS. By distinguishing between adrenal and ovarian sources, clinicians can tailor treatment strategies to address the underlying cause of androgen excess. For example, if the adrenal glands are identified as the primary source, treatment may focus on reducing adrenal androgen production through the use of glucocorticoids or other medications. Conversely, if the ovaries are the primary source, treatment may involve hormonal therapies aimed at reducing ovarian androgen production or improving insulin sensitivity.

In a discussion, study or a reading of the details, features, embodiments, aspects, and/or examples of the technology disclosed herein, any of the features, embodiments, aspects, and/or examples herein can be inter-combined (or inter-discussed) with the example details listed below:

Detail 1: A method for treating a condition and/or a sepsis in a subject in need thereof, the method comprising the steps of: (1) executing an analytical test on the subject, whereby the test will assess whether or not the subject is an appropriate candidate for a treatment by an administration of a therapeutically effective amount of an anti-androgen small molecule or a potential anti-androgen small molecule; (2) if the subject is an appropriate candidate after the test in step (1), administer the therapeutically effect amount of the anti-androgen small molecule and/or the potential anti-androgen small molecule to the subject; whereby the condition and/or the sepsis in the subject is improved compared to the same subject without the administering of the therapeutically effect amount of the anti-androgen small molecule and/or the potential anti-androgen small molecule to the subject.

Detail 2: The method of detail 1, further comprising the step of: (3) executing an analytical test to assess success of the administration of the anti-androgen small molecule or a potential anti-androgen small molecule, wherein the analytical test comprises an RNA sequencing test, a radiological test, a dexamethasone androgen suppression test, a COVID-19 test, a culture test, a nasopharyngeal viral clearance test, a protein expression test, a biomarker test, a clinical symptom assessment, or a combination thereof, and wherein the analytical test is performed at one or more time points after the administration; and/or determining a clinical score based on a physical examination for evaluation of signs of hyperandrogenic features such as evaluating body/face/scalp/pubic hair, oily skin, enlarged external genital (prostate/penis/clitoris); or a combination of clinical and laboratory criteria performed at any time. According to some aspects, the laboratory and/or clinical scores can be used for screening an individual in step 100, 102, and/or 104 of FIG. 1.

Detail 3: The method of detail 1, wherein the small molecule comprises sabizabulin, enzalutamide, apalutamide, darolutamide, relugolix, or a pharmaceutically acceptable hydrate, solvate, and/or salt form thereof, and wherein the small molecule is administered at a dose ranging from about 0.1 mg to about 1000 mg per day.

Detail 4: The method of detail 1, wherein the anti-androgen small molecule or the potential anti-androgen small molecule is administered in a therapeutically effective amount that is determined based on the severity and stage of the condition and/or sepsis in the subject, and wherein the therapeutically effective amount is adjusted during the course of treatment based on the subject's response.

Detail 5: The method of detail 1, wherein the improvement in the condition and/or sepsis is compared to the same subject without the administering of the therapeutically effective amount of the anti-androgen small molecule or the potential anti-androgen small molecule, and wherein the improvement is measured by one or more clinical parameters or biomarkers selected from the group consisting of body temperature, heart rate, respiratory rate, blood pressure, oxygen saturation, level of consciousness, urine output, blood cell counts, C-reactive protein levels, procalcitonin levels, cytokine levels, lactate levels, coagulation parameters, organ function markers, and mortality rate, and wherein the improvement is statistically significant.

Detail 6: The method of detail 1, wherein the anti-androgen small molecule or the potential anti-androgen small molecule is at least one of an anti-androgen small molecule or a potential anti-androgen small molecule that acts on the androgen receptor pathway, selected from the group consisting of androgen receptor antagonists, androgen synthesis inhibitors, androgen receptor degraders, selective androgen receptor modulators, and any combination thereof, and wherein the anti-androgen small molecule or potential anti-androgen small molecule has an IC50 or Ki value of less than 1 μM for the androgen receptor.

Detail 7: The method of detail 1, wherein the analytical test substantially assesses the appropriateness of the subject for the treatment by measuring one or more biomarkers, clinical symptoms, or risk factors associated with the condition and/or sepsis, and wherein the analytical test has a sensitivity and specificity of at least 80% for predicting the subject's response to the treatment.

Detail 8: The method of detail 1, wherein the administering is substantially of the therapeutically effective amount of the anti-androgen small molecule or the potential anti-androgen small molecule, and wherein the therapeutically effective amount is sufficient to improve the condition and/or sepsis in the subject, and wherein the therapeutically effective amount results in a plasma concentration of the anti-androgen small molecule or potential anti-androgen small molecule that is above the IC50 or Ki value for the androgen receptor for at least 50% of the dosing interval.

Detail 9: The method of detail 1, wherein the condition comprises COVID-19 infection, a traumatic lung injury, a skin condition, or any sign, symptom or condition that mimics a sepsis infection, including but not limited to pneumonia, acute respiratory distress syndrome, influenza, meningitis, encephalitis, pancreatitis, appendicitis, cholecystitis, pyelonephritis, cellulitis, necrotizing fasciitis, toxic shock syndrome, septic arthritis, osteomyelitis, endocarditis, and bacteremia, and wherein the condition is caused by a bacterial, viral, fungal, or parasitic pathogen.

Detail 10: The method of detail 1, wherein the condition and/or sepsis is one or more of a bacterial infection, viral infection, fungal infection, parasitic infection, inflammatory condition, or sepsis, and wherein the condition and/or sepsis is associated with an elevated level of androgen receptor activity in the subject.

Detail 11: The method of detail 1, wherein the subject is a human patient diagnosed with or at risk of developing the condition and/or sepsis, and wherein the subject is male or female and is of any age.

Detail 12: The method of detail 1, wherein the analytical test is performed prior to the administering and is used to select subjects who are likely to respond to the treatment, and wherein the analytical test is repeated during the course of treatment to monitor the subject's response.

Detail 13: The method of detail 1, wherein the administering is performed after the assessing and only in subjects who are determined to be appropriate candidates for the treatment based on the results of the analytical test, and wherein the administering is initiated within 48 hours of the onset of symptoms of the condition and/or sepsis.

Detail 14: The method of detail 1, wherein the improvement is assessed after the administering at one or more time points to monitor the effectiveness of the treatment, and wherein the improvement is assessed using one or more clinical parameters, biomarkers, or imaging modalities.

Detail 15: The method of detail 1, wherein the anti-androgen small molecule or the potential anti-androgen small molecule is administered orally, sublingually, buccally, intranasally, rectally, topically, transdermally, subcutaneously, intramuscularly, intravenously, intra-arterially, intrathecally, intraperitoneally, intravesically, intraocularly, intraarticularly, intracisternally, or by inhalation, using a suitable pharmaceutical formulation, and wherein the pharmaceutical formulation is a tablet, capsule, pill, powder, liquid, suspension, emulsion, gel, ointment, cream, patch, or aerosol.

Detail 16: The method of detail 1, wherein the anti-androgen small molecule or the potential anti-androgen small molecule is administered in a single dose or in multiple doses according to a therapeutic regimen that is tailored to the individual subject, and wherein the therapeutic regimen comprises administering the anti-androgen small molecule or potential anti-androgen small molecule once, twice, or three times daily for a period of at least one week.

Detail 17: The method of detail 1, wherein the therapeutically effective amount is determined based on the severity of the condition and/or sepsis in the subject, as well as other clinical factors such as age, weight, and overall health of the subject, and wherein the therapeutically effective amount is adjusted based on the subject's response to the treatment as assessed by one or more clinical parameters or biomarkers.

Detail 18: The method of detail 1, wherein the anti-androgen small molecule or the potential anti-androgen small molecule is selected based on the results of the analytical test, and wherein different anti-androgen small molecules or potential anti-androgen small molecules may be selected for different subjects depending on their individual test results, and wherein the anti-androgen small molecule or potential anti-androgen small molecule may be administered alone or in combination with one or more additional therapeutic agents, and wherein the one or more additional therapeutic agents are selected from the group consisting of antibiotics, antiviral agents, antifungal agents, anti-inflammatory agents, immunomodulatory agents, and supportive care agents.

Detail 19: The method of detail 1, wherein the analytical test assesses whether or not the subject is an appropriate candidate for the treatment by evaluating one or more genetic, biochemical, or clinical parameters that are predictive of the subject's response to the treatment, and wherein the one or more genetic, biochemical, or clinical parameters are selected from the group consisting of androgen receptor expression levels, androgen receptor mutation status, circulating androgen levels, inflammatory biomarkers, and clinical severity scores.

Detail 20: The method of detail 1, wherein the therapeutically effective amount is administered to the subject assessed as an appropriate candidate for the treatment, and wherein the subject is monitored for adverse reactions or side effects during the course of the treatment, and wherein the treatment is modified or discontinued if the subject experiences an adverse reaction or side effect that outweighs the potential benefit of the treatment.

Detail 21: A method for treating a condition and/or sepsis in a subject, the method comprising: executing a subject suitability test on the subject to assess whether the subject is an appropriate candidate for treatment by administration of a therapeutically effective amount of an androgen antagonist; and if the subject is an appropriate candidate based on the subject suitability test, administering the therapeutically effective amount of the androgen antagonist to the subject; whereby the condition and/or sepsis in the subject is improved compared to without the administering of the androgen antagonist.

Detail 22: The method of detail 21, wherein the androgen antagonist comprises an anti-androgen small molecule or a potential anti-androgen small molecule, and wherein the anti-androgen small molecule is selected from the group consisting of bicalutamide, enzalutamide, apalutamide, darolutamide, and combinations thereof.

Detail 23: The method of detail 21, wherein the subject suitability test is an analytical test comprising measuring the subject's serum testosterone levels, and wherein the subject is determined to be an appropriate candidate if the serum testosterone levels are above a predetermined threshold.

Detail 24: The method of detail 21, wherein the androgen antagonist is administered only if the subject is determined to be an appropriate candidate based on the subject suitability test, and wherein the androgen antagonist is administered orally, intravenously, intramuscularly, or subcutaneously.

Detail 25: The method of detail 21, wherein the condition is sepsis caused by a gram-negative bacterial infection, and wherein the gram-negative bacterial infection is caused by Escherichia coli, Pseudomonas aeruginosa, Klebsiella pneumoniae, or combinations thereof.

Detail 26: The method of detail 21, wherein the therapeutically effective amount of the androgen antagonist improves the condition and/or sepsis in the subject compared to the same subject without the administration of the androgen antagonist, and wherein the improvement is measured by a reduction in the subject's Sequential Organ Failure Assessment (SOFA) score.

Detail 27: A method for determining if a subject is an appropriate candidate for treatment of a condition and/or sepsis by administration of an androgen antagonist, the method comprising: executing a subject suitability test on the subject to assess whether the subject is the appropriate candidate for the treatment by the administration of a therapeutically effective amount of the androgen antagonist; wherein if the subject is the appropriate candidate based on the subject suitability test, the therapeutically effective amount of the androgen antagonist is administered to the subject to improve the condition and/or sepsis compared to without the administration of the androgen antagonist.

Detail 28: The method of detail 27, wherein the androgen antagonist comprises at least one of an anti-androgen small molecule or a potential anti-androgen small molecule, and wherein the potential anti-androgen small molecule is a compound that exhibits an IC50 value of less than 1 μM in an in vitro androgen receptor binding assay.

Detail 29: The method of detail 27, wherein the subject suitability test comprises an analytical test that measures the subject's serum levels of interleukin-6 (IL-6), and wherein the subject is determined to be an appropriate candidate if the serum IL-6 levels are above a predetermined threshold.

Detail 30: The method of detail 27, wherein the condition is sepsis caused by a fungal infection, and wherein the fungal infection is caused by Candida albicans, Aspergillus fumigatus, or combinations thereof.

Detail 31: A system for treating a condition and/or sepsis in a subject, the system comprising: means for executing a subject suitability test on the subject to assess whether the subject is an appropriate candidate for treatment by administration of a therapeutically effective amount of an androgen antagonist; and means for administering the therapeutically effective amount of the androgen antagonist to the subject if the subject is an appropriate candidate based on the subject suitability test; whereby the condition and/or sepsis in the subject is improved compared to without the administering of the androgen antagonist.

Detail 32: The system of detail 31, wherein the androgen antagonist comprises an anti-androgen small molecule or a potential anti-androgen small molecule, and wherein the anti-androgen small molecule is formulated as a sustained-release composition for parenteral administration.

Detail 33: The system of detail 31, wherein the subject suitability test is an analytical test comprising measuring the subject's serum levels of procalcitonin (PCT), and wherein the subject is determined to be an appropriate candidate if the serum PCT levels are above a predetermined threshold.

Detail 34: The system of detail 31, wherein the androgen antagonist is administered only if the subject is determined to be an appropriate candidate based on the subject suitability test, and wherein the androgen antagonist is administered in combination with an antibiotic or an antifungal agent.

Detail 35: The system of detail 31, wherein the condition is sepsis caused by a viral infection, and wherein the viral infection is caused by influenza virus, respiratory syncytial virus (RSV), or combinations thereof.

Detail 36: The system of detail 31, wherein the therapeutically effective amount of the androgen antagonist improves the condition and/or sepsis in the subject compared to the same subject without the administration of the androgen antagonist, and wherein the improvement is measured by a reduction in the subject's Acute Physiology and Chronic Health Evaluation (APACHE) II score.

Detail 37: A system for determining if a subject is an appropriate candidate for treatment of a condition and/or sepsis by administration of an androgen antagonist, the system comprising: means for executing a subject suitability test on the subject to assess whether the subject is the appropriate candidate for the treatment by the administration of a therapeutically effective amount of the androgen antagonist; wherein if the subject is the appropriate candidate based on the subject suitability test, the therapeutically effective amount of the androgen antagonist is administered to the subject to improve the condition and/or sepsis compared to without the administration of the androgen antagonist.

Detail 38: The system of detail 37, wherein the androgen antagonist comprises at least one of an anti-androgen small molecule or a potential anti-androgen small molecule, and wherein the potential anti-androgen small molecule is a natural product or a derivative thereof.

Detail 39: The system of detail 37, wherein the subject suitability test comprises an analytical test that measures the subject's serum levels of C-reactive protein (CRP), and wherein the subject is determined to be an appropriate candidate if the serum CRP levels are above a predetermined threshold.

Detail 40: The system of detail 37, wherein the condition is sepsis caused by a parasitic infection, and wherein the parasitic infection is caused by Plasmodium falciparum, Toxoplasma gondii, or combinations thereof.

Detail 41: A method for treating a condition and/or a sepsis in a subject in need thereof, the method comprising the steps of: (1) executing an analytical test on the subject, whereby the test will assess whether or not the subject is an appropriate candidate for a treatment by an administration of a therapeutically effective amount of an anti-androgen small molecule or a potential anti-androgen small molecule; (2) if the subject is an appropriate candidate after the test in step (1), administer the therapeutically effect amount of the anti-androgen small molecule and/or the potential anti-androgen small molecule to the subject; whereby the condition and/or the sepsis in the subject is improved compared to the same subject without the administering of the therapeutically effect amount of the anti-androgen small molecule and/or the potential anti-androgen small molecule to the subject.

Detail 42: The method of detail 41, further comprising the step of: (3) executing an analytical test to assess success of the administration of the anti-androgen small molecule or a potential anti-androgen small molecule, wherein the analytical test comprises an RNA sequencing test, a radiological test, a dexamethasone androgen suppression test, a COVID-19 test, a culture test, a nasopharyngeal viral clearance test, a protein expression test, a biomarker test, a clinical symptom assessment, or a combination thereof, and wherein the analytical test is performed at one or more time points after the administration, and wherein the analytical test is used to guide further treatment decisions such as adjusting the dose, frequency, or duration of administration of the anti-androgen small molecule or potential anti-androgen small molecule, or switching to a different anti-androgen small molecule or potential anti-androgen small molecule; and/or determining a clinical score based on a physical examination for evaluation of signs of hyperandrogenic features such as evaluating body/face/scalp/pubic hair, oily skin, enlarged external genital (prostate/penis/clitoris); or a combination of clinical and laboratory criteria performed at any time. According to some aspects, the laboratory and/or clinical scores can be used for screening an individual in step 100, 102, and/or 104 of FIG. 1.

Detail 43: The method of detail 41, wherein the small molecule comprises sabizabulin, enzalutamide, apalutamide, darolutamide, relugolix, or a pharmaceutically acceptable hydrate, solvate, and/or salt form thereof, and wherein the small molecule is administered at a dose ranging from about 0.1 mg to about 1000 mg per day, and wherein the small molecule is administered in a pharmaceutical composition comprising one or more pharmaceutically acceptable carriers, excipients, or diluents.

Detail 44: The method of detail 41, wherein the anti-androgen small molecule or the potential anti-androgen small molecule is administered in a therapeutically effective amount that is determined based on the severity and stage of the condition and/or sepsis in the subject, and wherein the therapeutically effective amount is adjusted during the course of treatment based on the subject's response, and wherein the therapeutically effective amount is administered in divided doses or in a single daily dose, and wherein the therapeutically effective amount is administered for a duration of at least one week, at least two weeks, at least one month, at least two months, at least three months, at least six months, or at least one year.

Detail 45: The method of detail 41, wherein the improvement in the condition and/or sepsis is compared to the same subject without the administering of the therapeutically effective amount of the anti-androgen small molecule or the potential anti-androgen small molecule, and wherein the improvement is measured by one or more clinical parameters or biomarkers selected from the group consisting of body temperature, heart rate, respiratory rate, blood pressure, oxygen saturation, level of consciousness, urine output, blood cell counts, C-reactive protein levels, procalcitonin levels, cytokine levels, lactate levels, coagulation parameters, organ function markers, and mortality rate, and wherein the improvement is statistically significant, and wherein the improvement is maintained for at least one week, at least two weeks, at least one month, at least two months, at least three months, at least six months, or at least one year after the administration.

Detail 46: The method of detail 41, wherein the anti-androgen small molecule or the potential anti-androgen small molecule is at least one of an anti-androgen small molecule or a potential anti-androgen small molecule that acts on the androgen receptor pathway, selected from the group consisting of androgen receptor antagonists, androgen synthesis inhibitors, androgen receptor degraders, selective androgen receptor modulators, and any combination thereof, and wherein the anti-androgen small molecule or potential anti-androgen small molecule has an IC50 or Ki value of less than 1 μM for the androgen receptor, and wherein the anti-androgen small molecule or potential anti-androgen small molecule has a selectivity for the androgen receptor over other steroid hormone receptors of at least 10-fold, at least 50-fold, at least 100-fold, at least 500-fold, or at least 1000-fold.

Detail 47: The method of detail 41, wherein the analytical test substantially assesses the appropriateness of the subject for the treatment by measuring one or more biomarkers, clinical symptoms, or risk factors associated with the condition and/or sepsis, and wherein the analytical test has a sensitivity and specificity of at least 80% for predicting the subject's response to the treatment, and wherein the analytical test is a gene expression assay, a protein expression assay, a metabolomics assay, a histological assay, a blood chemistry assay, a clinical symptom assessment, or a combination thereof.

Detail 48: The method of detail 41, wherein the administering is substantially of the therapeutically effective amount of the anti-androgen small molecule or the potential anti-androgen small molecule, and wherein the therapeutically effective amount is sufficient to improve the condition and/or sepsis in the subject, and wherein the therapeutically effective amount results in a plasma concentration of the anti-androgen small molecule or potential anti-androgen small molecule that is above the IC50 or Ki value for the androgen receptor for at least 50% of the dosing interval, and wherein the therapeutically effective amount results in a steady-state plasma concentration of the anti-androgen small molecule or potential anti-androgen small molecule that is between 0.1 nM and 10 μM.

Detail 49: The method of detail 41, wherein the condition comprises COVID-19 infection, a traumatic lung injury, a skin condition, or any sign, symptom or condition that mimics a sepsis infection, including but not limited to pneumonia, acute respiratory distress syndrome, influenza, meningitis, encephalitis, pancreatitis, appendicitis, cholecystitis, pyelonephritis, cellulitis, necrotizing fasciitis, toxic shock syndrome, septic arthritis, osteomyelitis, endocarditis, and bacteremia, and wherein the condition is caused by a bacterial, viral, fungal, or parasitic pathogen, and wherein the condition is associated with an elevated level of androgen receptor activity in the affected tissue or organ.

Detail 50: The method of detail 41, wherein the condition and/or sepsis is one or more of a bacterial infection, viral infection, fungal infection, parasitic infection, inflammatory condition, or sepsis, and wherein the condition and/or sepsis is associated with an elevated level of androgen receptor activity in the subject, and wherein the elevated level of androgen receptor activity is determined by measuring the expression level of the androgen receptor gene or protein in a biological sample from the subject, or by measuring the level of one or more downstream targets of the androgen receptor in a biological sample from the subject.

Detail 51: The method of detail 41, wherein the subject is a human patient diagnosed with or at risk of developing the condition and/or sepsis, and wherein the subject is male or female and is of any age, and wherein the subject has one or more risk factors for developing the condition and/or sepsis, including but not limited to advanced age, chronic illness, immunosuppression, surgery, trauma, burns, indwelling medical devices, prolonged hospitalization, residence in a long-term care facility, and exposure to infectious agents, and wherein the subject has one or more comorbidities that may affect the severity or outcome of the condition and/or sepsis, including but not limited to diabetes, cardiovascular disease, lung disease, liver disease, kidney disease, cancer, and neurological disorders.

Detail 52: The method of detail 41, wherein the analytical test is performed prior to the administering and is used to select subjects who are likely to respond to the treatment, and wherein the analytical test is repeated during the course of treatment to monitor the subject's response, and wherein the results of the analytical test are used to guide decisions regarding the continuation, modification, or discontinuation of treatment, and wherein the analytical test is performed using a biological sample obtained from the subject, such as blood, serum, plasma, urine, saliva, sputum, cerebrospinal fluid, or tissue, and wherein the biological sample is obtained by venipuncture, finger stick, lumbar puncture, bronchoscopy, or biopsy.

Detail 53: The method of detail 41, wherein the administering is performed after the assessing and only in subjects who are determined to be appropriate candidates for the treatment based on the results of the analytical test, and wherein the administering is initiated within 48 hours of the onset of symptoms of the condition and/or sepsis, and wherein the administering is performed in a hospital, clinic, or outpatient setting, and wherein the administering is performed by a healthcare professional such as a physician, nurse, or pharmacist, and wherein the administering is performed using standard techniques and equipment for the selected route of administration.

Detail 54: The method of detail 41, wherein the improvement is assessed after the administering at one or more time points to monitor the effectiveness of the treatment, and wherein the improvement is assessed using one or more clinical parameters, biomarkers, or imaging modalities, and wherein the clinical parameters include vital signs, physical examination findings, and laboratory test results, and wherein the biomarkers include markers of inflammation, infection, organ dysfunction, and coagulation, and wherein the imaging modalities include radiography, ultrasound, computed tomography, magnetic resonance imaging, and nuclear medicine imaging, and wherein the improvement is assessed by comparing the subject's clinical status before and after treatment, or by comparing the subject's clinical status to that of a control group that did not receive the treatment.

Detail 55: The method of detail 41, wherein the anti-androgen small molecule or the potential anti-androgen small molecule is administered orally, sublingually, buccally, intranasally, rectally, topically, transdermally, subcutaneously, intramuscularly, intravenously, intra-arterially, intrathecally, intraperitoneally, intravesically, intraocularly, intraarticularly, intracisternally, or by inhalation, using a suitable pharmaceutical formulation, and wherein the pharmaceutical formulation is a tablet, capsule, pill, powder, liquid, suspension, emulsion, gel, ointment, cream, patch, or aerosol, and wherein the pharmaceutical formulation is designed to provide immediate release, sustained release, controlled release, or targeted delivery of the anti-androgen small molecule or potential anti-androgen small molecule, and wherein the pharmaceutical formulation is prepared using standard techniques and equipment for pharmaceutical manufacturing and quality control.

Detail 56: The method of detail 41, wherein the anti-androgen small molecule or the potential anti-androgen small molecule is administered in a single dose or in multiple doses according to a therapeutic regimen that is tailored to the individual subject, and wherein the therapeutic regimen comprises administering the anti-androgen small molecule or potential anti-androgen small molecule once, twice, or three times daily for a period of at least one week, and wherein the therapeutic regimen is designed to achieve and maintain a steady-state plasma concentration of the anti-androgen small molecule or potential anti-androgen small molecule that is within the therapeutic range, and wherein the therapeutic regimen is adjusted as needed based on the subject's response to treatment, as assessed by clinical parameters, biomarkers, or imaging studies, and wherein the therapeutic regimen is continued until the subject achieves a satisfactory clinical response or experiences unacceptable adverse effects.

Detail 57: The method of detail 41, wherein the therapeutically effective amount is determined based on the severity of the condition and/or sepsis in the subject, as well as other clinical factors such as age, weight, and overall health of the subject, and wherein the therapeutically effective amount is adjusted based on the subject's response to the treatment as assessed by one or more clinical parameters or biomarkers, and wherein the therapeutically effective amount is selected to provide a plasma concentration of the anti-androgen small molecule or potential anti-androgen small molecule that is above the minimum effective concentration and below the maximum tolerated concentration, and wherein the therapeutically effective amount is calculated using pharmacokinetic and pharmacodynamic modeling and simulation based on preclinical and clinical data.

Detail 58: The method of detail 41, wherein the anti-androgen small molecule or the potential anti-androgen small molecule is selected based on the results of the analytical test, and wherein different anti-androgen small molecules or potential anti-androgen small molecules may be selected for different subjects depending on their individual test results, and wherein the anti-androgen small molecule or potential anti-androgen small molecule may be administered alone or in combination with one or more additional therapeutic agents, and wherein the one or more additional therapeutic agents are selected from the group consisting of antibiotics, antiviral agents, antifungal agents, anti-inflammatory agents, immunomodulatory agents, and supportive care agents, and wherein the one or more additional therapeutic agents are selected based on the specific pathogen or pathogenic mechanism involved in the subject's condition and/or sepsis, and wherein the one or more additional therapeutic agents are administered simultaneously, sequentially, or alternately with the anti-androgen small molecule or potential anti-androgen small molecule, and wherein the one or more additional therapeutic agents are administered by the same or different routes as the anti-androgen small molecule or potential anti-androgen small molecule.

Detail 59: The method of detail 41, wherein the analytical test assesses whether or not the subject is an appropriate candidate for the treatment by evaluating one or more genetic, biochemical, or clinical parameters that are predictive of the subject's response to the treatment, and wherein the one or more genetic, biochemical, or clinical parameters are selected from the group consisting of androgen receptor expression levels, androgen receptor mutation status, circulating androgen levels, inflammatory biomarkers, and clinical severity scores, and wherein the androgen receptor expression levels are measured by quantitative reverse transcription polymerase chain reaction, Western blotting, immunohistochemistry, or flow cytometry, and wherein the androgen receptor mutation status is determined by DNA sequencing, allele-specific polymerase chain reaction, or restriction fragment length polymorphism analysis, and wherein the circulating androgen levels are measured by enzyme-linked immunosorbent assay, radioimmunoassay, or liquid chromatography-tandem mass spectrometry, and wherein the inflammatory biomarkers are measured by enzyme-linked immunosorbent assay, multiplex immunoassay, or high-sensitivity C-reactive protein assay, and wherein the clinical severity scores are calculated using validated scoring systems such as the Acute Physiology and Chronic Health Evaluation II score, the Sequential Organ Failure Assessment score, or the quick Sequential Organ Failure Assessment score.

Detail 60: The method of detail 41, wherein the therapeutically effective amount is administered to the subject assessed as an appropriate candidate for the treatment, and wherein the subject is monitored for adverse reactions or side effects during the course of the treatment, and wherein the treatment is modified or discontinued if the subject experiences an adverse reaction or side effect that outweighs the potential benefit of the treatment, and wherein the adverse reactions or side effects may include nausea, vomiting, diarrhea, constipation, abdominal pain, headache, dizziness, fatigue, insomnia, anxiety, depression, hot flashes, gynecomastia, decreased libido, erectile dysfunction, osteoporosis, cardiovascular events, hepatotoxicity, or dermatological reactions, and wherein the adverse reactions or side effects are managed by adjusting the dose or frequency of administration of the anti-androgen small molecule or potential anti-androgen small molecule, by administering concomitant medications to treat the adverse reactions or side effects, or by discontinuing the anti-androgen small molecule or potential anti-androgen small molecule and switching to an alternative treatment.

In some embodiments, the invention is used for the treatment of a subject or patient who suffers from traumatic injury, such as traumatic lung injury, such as a traumatic lung injury that coincides with a COVID-19 infection.

In some embodiments, the antiandrogen is sabizabulin. In another embodiment, the therapeutically similar antiandrogen is or comprises an antiandrogen selected from the group consisting of proxalutamide, enzalutamide, and bicalutamide. The method can also include administering dutasteride or spironolactone to treat sepsis and conditions that mimic sepsis. For example, at present, two molecules showed benefit vs “standard-of-care” which included steroids for patients with severe lung involvement, the antiandrogens proxalutamide and sabizabulin.

In some embodiments, the analytical test to assess whether the subject or patient is an appropriate candidate for treatment by the administration of sabizabulin or a therapeutically similar antiandrogen is by RNA sequencing to provide a diagnostic for sepsis that can also monitor the indicia of treatment and recovery (bacterial counts reduce, physiology returns to steady-state). See Intl. Pat Publ. WO 2021/163692 and U.S. Pat. Publ. US 2022/0340972. This technology can be used for many other hospital conditions, particularly those needing an intensive care unit stay with the attendant risk of bacterial infection, such as trauma, stroke, myocardial infarction, or major surgery. This technology provides an initial diagnostic for sepsis that can also monitor the indicia of treatment and recovery (bacterial counts reduce, physiology returns to steady-state). The invention can be used for many other hospital conditions, particularly those needing an intensive care unit stay with the attendant risk of bacterial infection, such as trauma, stroke, myocardial infarction, or major surgery.

According to some aspects, the analytical test to assess whether the subject or patient is an appropriate candidate for treatment by the administration of sabizabulin or a therapeutically similar antiandrogen can be supplemented by other standard tests.

The analytical test to assess the progress or success of the administration of sabizabulin or therapeutically similar antiandrogen can be an RNA sequencing test to assess lung improvement. See Intl. Pat. Publ. WO 2022/225960.

In some embodiments, the analytical test to assess the progress or success of the administration of sabizabulin or therapeutically similar antiandrogen is a radiological test to assess lung improvement in patients with severe COVID-19. The radiological changes follow the improvement in clinical outcomes with proxalutamide. X-ray scans are informative lung assessments. Computed tomography (CT) scans are more informative than X-ray scans.

In the clinical trial in Brazil (discussed herein), the trial of proxalutamide in Brazil was with both arms using corticosteroids, either dexamethasone or methylprednisolone. So, the patients that received proxalutamide had proxalutamide+corticosteroids. Because corticosteroids were part of standard-of-care. In another embodiment, the analytical test to assess whether the subject or patient is an appropriate candidate for treatment by the administration of sabizabulin or a therapeutically similar antiandrogen can be supplemented by the current standard of care for the treatment of sepsis, which is the use of systemic steroids, maintaining vital signs, palliative support, and treating infections with antibiotics. The current standard of care for COVID-19 (the vaccine strategy being a method of prevention rather than a method of treatment) includes the administration of dexamethasone, then the administration of remdesivir, and then the administration of tocilizumab, baricitinib, or tofacitinib. Dexamethasone also has known to have mild antiandrogen effects, by reducing the expression of adrenal corticosteroids. The combination of proxalutamide, sabizabulin, and dexamethasone (or association of more potent anti androgens with corticosteroids) is also used. The clinical effects of dexamethasone on androgen expression have been studied, for example, at the following link: linkinghub.elsevier.com/retrieve/pii/S0015028298000612. There is a test called “dexamethasone androgen suppression test”. Which reduces more DHEAS than Testosterone . . . Particularly in cases of elevated DHEAS (adrenal androgen), for example, see: academic.oup.com/humrep/article/26/11/3138/657817, for example, at FIG. 1. It is important to note that the relationships of synthetic corticosteroids with Androgen Receptors are complex and are characterized by a ‘widely known minor antiandrogen effect of corticosteroid administration by suppression of androgens’.

An unexpected result of anti-androgen therapy in cases of sepsis or toxic shock that present in the hospital with lung involvement documented by computed tomography (CT) scans is the fast resolution of the radiographic images, compared to standard care.

In one aspect, the invention provides an antiandrogen therapy (antiandrogen steroid approach) to substitute for the current Corticosteroid therapy (hormonal adrenal steroid approach).

In another aspect, the therapy is an improvement on prior methods of medical management because there are no medications besides antimicrobials for the treatment of sepsis, and antimicrobials are not useful for the virally-caused conditions that mimic sepsis, such as COVID-19 infections.

In another aspect, the therapy can begin even in a chronic inflammation state. The therapy does not need to be started early, when the virus is circulating The therapy still reverts sepsis and inflammation despite timing.

In another aspect, the invention provides that sabizabulin's beneficial effect is due to a general improvement in sepsis induced by SARS-CoV-2.

In yet another aspect, the invention provides that the administration of anti-androgens are anti-fibrotic. This aspect advantageously provides a benefit for the treatment of acute respiratory distress syndrome (ARDS). The invention provides a way for medical providers to initiate a therapeutic treatment as soon as they can observe a need, e.g., in a computed tomography (CT) scan, for respiratory support for a subject or patient who has sepsis with lung involvement, thus improving clinical outcomes and preventing lung fibrosis.

Any detail, embodiment, example, and/or feature can be provided in a kit form. The technology herein solves a long-felt but unmet need in the face of years of failure by others. The methods herein can be applied prophylactically to a normal healthy subject. A subject can be one who has been previously diagnosed with or identified as suffering from or having a condition in need of treatment (e.g., an infection) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition, or one or more complications related to a condition or a subject who does not exhibit risk factors. A “subject in need” of treatment for a particular condition can be a subject having that condition, diagnosed as having that condition, or at risk of developing that condition.

Some aspects, modes, embodiments, variations, and features of the invention are described below in various levels of detail to provide a substantial understanding of the invention.

Industrial applicability: The invention is useful for the treatment of sepsis, which is a life-threatening organ dysfunction due to a dysregulated host response to infection. Sabizabulin and therapeutically similar antiandrogen medications are advantageously lifesaving to a broad group of patients presenting with sepsis from any type of infectious etiology, along with patients that have sepsis-like conditions that include hemorrhagic shock and trauma.

Despite declining age-standardized incidence and mortality, sepsis remains a significant cause of health loss worldwide. Rudd et al., The Lancet, 395(10219), 200-211 (Jan. 18, 2020). Sepsis is treatable, and timely implementation of targeted interventions improves outcomes. The current standard of care for the treatment of sepsis is the use of systemic steroids, maintaining vital signs, palliative support, and treating infections with antibiotics.

Sepsis is diagnosed clinically by the presence of acute infection and organ dysfunction. Singer et al., JAMA, 315, 801-810 (February 2016). Unlike the previous concepts of septicemia or blood poisoning, the definition focuses on the host response as the major source of morbidity and mortality. Bone et al., Chest, 101, 1644-1655 (1992). Sepsis results from an underlying infection, so sepsis is an intermediate cause of health loss. Because, according to the principles of the International Classification of Diseases (ICD), causes of death are assigned based on the underlying disorder that triggers the chain of events leading to death rather than intermediate causes, sepsis, when reported as the cause of death, are considered miscoded.

Physicians diagnose sepsis using clinical judgment under one or more clinical scores. The systemic inflammatory response syndrome (SIRS) approach assesses an inflammatory state affecting the whole body, which is the body's response to an infectious or non-infectious challenge. Jui et al., American College of Emergency Physicians), Ch. 146, Septic Shock. in Tintinalli's Emergency Medicine, A Comprehensive Study Guide, 7th edition, (New York, McGraw-Hill, 2011). pp. 1003-14. Sepsis has both pro-inflammatory and anti-inflammatory components. The qSOFA approach simplifies the SOFA score by including only its three clinical criteria and by including any altered mentation. Singer et al., JAMA, 315, 801-810 (February 2016). qSOFA can easily and quickly be repeated serially on patients.

Sepsis patients experience consumptive thrombocytopenia, hemolytic anemia, vascular microthrombosis, multi-organ dysfunction syndrome, coagulopathy, septic shock, respiratory failure, fever, leukopenia, hypotension, leukocytosis, high cytokine production and high predisposition to opportunistic infections. Olwal et al., Front. Immunol. (February 2021). Sepsis happens when an infection causes your body's immune system to mount an extreme response. This excessive response can lead to tissue damage, organ failure, and even death if not treated promptly. As a viral infection, COVID-19 can lead to sepsis. People with severe COVID-19 may experience symptoms such as fever and chills, difficulty breathing, pain or discomfort, and confusion. These are also common signs of sepsis. See Based on Science, What is the connection between COVID-19 and sepsis? National Academies (Feb. 14, 2022).

Treatment rationale: The inventors conceived of the therapeutic method of administration when considering the compounds that were proposed for the improvement of sepsis outcomes. One class of compounds were the statins, e.g., atorvastatin. See BMJ, 11, 57 (2013). Another compound was metformin. Both have mild antiandrogen activity in addition to their main clinical activity. The inventors conceived that their antiandrogenic activity could be a common link with the improvement of sepsis outcomes. The inventors also noted that fluvoxamine also has antiandrogenic activity and anti-sepsis activity in a fecal peritonitis model.

The inventors developed this plausible essential role of androgen receptor for SARS-CoV-2 infection, based on the androgen receptor activation for the transcription of transmembrane protease, serine 2 (TMPRSS2). In a study of androgen-stimulated prostate cancer cells (LNCaP), TMPRSS2 mRNA expression increase was mediated by the androgen receptor. The ACE2 receptor, also critical for SARS-CoV-2 viral infectivity, is affected by male sex hormones with higher activity found in males. This mechanism could explain why males seem to be more vulnerable, and why children are more resistant to infections before adrenarche and puberty. Androgen receptor gene polymorphisms were linked with other known risk factors such as hypertension and possibly ethnicity. See Wambier et al., Drug Dev Res., 81(7), 771-776 (2020). Many are the conditions that could increase androgen activity in females, increasing vulnerability to COVID-19. Within the same age group, females generally have much lower levels of testosterone than males. Ober et al. (2008). Monthly fluctuations in androgen hormones occur during the menstrual cycle. Congenital adrenal hyperplasia (CAH) is a class of autosomal recessive disorders characterized by a specific hormone deficiency referred to as 21-hydroxylase deficiency. 21-hydroxylase deficiency results in excess adrenal precursors which are excessively metabolized into androgens i.e., testosterone and dihydrotestosterone (DHT). White & Speiser (2000). Metabolism has a direct relationship with hyperandrogenism in females, genetically higher testosterone levels in females was associated with increased risk of type 2 diabetes. The role of androgens on COVID-19 disease severity and mortality could explain the gender bias in mortality rates. In addition, the androgen mechanism explains the low rate of mortality among pre-pubescent children.

During the continuing SARS-CoV-2 (COVID-19) pandemic, several studies reported a significant difference in the rate of severe cases between adult females and adult males (42% vs 58%). Among children under the age of fourteen, the rate of severe cases was low. Past investigation on male androgenetic alopecia (AGA) led the investigators to investigate an association between androgens and COVID-19 pathogenesis. In normal subjects, androgen expression demonstrates significant variation between men and women and between adults and pre-pubescent children.

In a published proxalutamide study, the inventors found no difference in survival when initiated in patients that don't require 02. The study only included patients that required 02. They clearly showed efficacy. All antiandrogens reduced mortality with lung involvement when initiated on patients that require 02.

Sabizabulin was tested on hospitalized patients requiring 02 who saw reduction on mortality, and better in-hospital outcomes. Many of the patients were at great risk for sepsis and shock. Veru, Inc. completed a positive Phase 3 COVID-19 study evaluating sabizabulin in hospitalized moderate-to-severe COVID-19 patients (WHO 4-supplemental oxygen) at high risk for acute respiratory distress syndrome. The primary endpoint was the proportion of deaths by Day 60. See the Phase 3 COVID-19 trial results evaluating the efficacy and safety of oral sabizabulin in The New England Journal of Medicine (2020).

A mortality reduction among patients requiring oxygen at baseline is consistent with results from a larger randomized, double-blind, placebo-controlled study of androgen receptor blockade. For example, only two molecules, both antiandrogens, showed benefit vs “standard-of-care” which includes steroids for patients with severe lung involvement, proxalutamide and sabizabulin.

Additional Definitions: for convenience, the meaning of some terms and phrases used in the specification, examples, and appended claims, can be considered below. Unless stated otherwise or implicit from context, these terms and phrases shall have the meanings herein.

5-alpha-reductase inhibitor (5ARi) has the biomedical art-recognized meaning. Dutasteride and finasteride are 5-alpha-reductase inhibitors. 5-alpha reductase inhibitors such as dutasteride and finasteride are prescribed for hyperandrogenic features involving dihydrotestosterone activity, such as labeled and off-label use in dermatology to treat AGA and the labeled indication use in urology for benign prostatic hyperplasia. See United States Food & Drug Administration. Avodart® (dutasteride) Soft Gelatin Capsules. Highlights of Prescribing Information (2008). One limitation of SARis is the time course required to achieve systemic DHT reductions.

Acute respiratory distress syndrome (ARDS) has the biomedical art-defined meaning. Acute respiratory distress syndrome is a type of respiratory failure characterized by rapid onset of widespread inflammation in the lungs. Symptoms include shortness of breath, rapid breathing, and bluish skin coloration. A main cause of ARDS is sepsis.

Androgen receptor (AR) has the biomedical art-recognized meaning. Antiandrogen has the biomedical art-recognized meaning. Androgen receptor antagonists include but are not limited to flutamide, hydroxyflutamide, bicalutamide, enzalutamide, and apalutamide.

Clinical trial has meaning defined by the United States Food & Drug Administration (FDA).

Comprises and comprising refer to elements, components, or steps in a non-exclusive manner, indicating that the referenced elements, components, or steps may be present, used, or combined with other elements, components, or steps. The singular terms a, an, and the include plural referents unless context indicates otherwise. Similarly, the inclusive term or should cover the term and unless the context indicates otherwise. The abbreviation e.g., means a non-limiting example and is synonymous with the term for example.

Computed tomography (CT) scan has the biomedical art-recognized meaning. CT scanning has several advantages over traditional two-dimensional medical radiography. First, CT eliminates the superimposition of images of structures outside the area of interest. Second, CT scans have greater image resolution, enabling examination of finer details. Mikla & Mikla, Medical Imaging Technology (Elsevier, Aug. 23, 2013).

Degarelix (Firmagon®) has the biomedical art-recognized meaning. Degarelix is a gonadotropin-releasing hormone (GnRH) receptor antagonist. The United States Veteran's Administration instituted a clinical trial (HITCH) for COVID-19. See Nickols et al., Effect of androgen suppression on clinical outcomes in hospitalized men with COVID-19, The HITCH Randomized Clinical Trial. JAMA Networking Open., 5(4), e227852 (Apr. 1, 2022).

Dihydrotestosterone (DHT) promotes the expression of transmembrane serine protease 2 (TMPRSS2), and androgen signaling regulates SARS-CoV-2 infectivity. Cadegiani et al., BMJ Case Reports, 14, e241572 (2021).

Diarylhydantoin compound has the biomedical art-recognized meaning, and includes among other chemical compounds: flutamide, bicalutamide, enzalutamide, apalutamide (ERLEADA™), and proxalutamide. These compounds competitively inhibit androgen binding, block androgen receptor nuclear translocation, and prevent their binding to DNA. Tran et al., Science, 324(5928), 787-790 (2009).

Enzalutamide (XTANDI®) has the biomedical art-recognized meaning. The principal argument against using enzalutamide is the decision from the data safety monitoring board (DSMB) decision. The trial was stopped early, but conclusions were based on key, uneven distributions between groups. Welen et al. A phase 2 trial of the effect of antiandrogen therapy on COVID-19 outcome: no evidence of benefit, supported by epidemiology and in vitro data. Eur. Urol., 81, 285-93 (2022).

Finasteride has the biomedical art-recognized meaning of a 5-α reductase inhibitor widely used to manage benign prostate hyperplasia and male pattern hair loss. Finasteride use is associated with a clinically and statistically significant improvement in peripheral capillary oxygen saturation at five days of therapy. Zarehoseinzade et al., Finasteride in hospitalized adult males with COVID-19: A risk factor for severity of the disease or an adjunct treatment: A randomized controlled clinical trial TT. MJIRI, 35(1), 232-237 (2021). Finasteride has a very short half-life of six hours, compared to five weeks for dutasteride. See Wambier et al., Brazilian blood donation eligibility criteria for dermatologic patients. An. Bras. Dermatol., 87(4), 590-595 (2012).

Inflammation has the biomedical art-recognized meaning. Keloid has the biomedical art-recognized meaning.

Mann Whitney U tests have the statistical art-recognized meaning. The Mann-Whitney U test (also called the Mann-Whitney-Wilcoxon (MWW), Wilcoxon rank-sum test, or Wilcoxon-Mann-Whitney test) is a nonparametric test of the null hypothesis that it is equally likely that a randomly selected value from one population is less than or greater than a randomly selected value from a second population. This test can investigate whether two independent samples were selected from populations having the same distribution.

Next Generation Sequencing (NGS) has the biomedical art-recognized meaning. NGS technology is typically highly scalable, allowing the entire genome to be sequenced at once. Usually, this is accomplished by fragmenting the genome into small pieces, randomly sampling for a fragment, and sequencing it using various technologies.

Nonsteroidal antiandrogen (NSAA) has the biomedical art-recognized meaning. First-generation oral nonsteroidal antiandrogens such as bicalutamide, flutamide, and nilutamide that compete with DHT for binding androgen receptor are associated with a moderate rise in testosterone. Next-generation orally available nonsteroidal androgen receptor inhibitors enzalutamide (XTANDI®), apalutamide (ERLEADA™) and darolutamide (NUBEQA™) competitively bind the ligand-binding domain of the androgen receptor to limit nuclear translocation and downstream signaling

Pandemic has the biomedical art-recognized meaning. Placebo has the biomedical art-recognized meaning.

Principal Component Analysis (PCA) has the biomedical art-recognized meaning. The principal component analysis is a statistical procedure that uses an orthogonal transformation to convert a set of observations of possibly correlated variables (entities, each of which takes on various numerical values) into a set of values of linearly uncorrelated variables called principal components.

Proxalutamide (GT0918) is a second-generation nonsteroidal androgen receptor antagonist that is more potent than other antiandrogen compounds. See Qu et al., Invest. New Drugs, 38(5), 1292-1302 (2020). Clinical evidence demonstrated that proxalutamide lowers androgen receptor expression and activity. Proxalutamide lowers the expression of ACE2. Proxalutamide demonstrates a dual mechanism of action. Proxalutamid inhibits androgen receptor as well as exhibiting pharmacological effects of inducing the down-regulation of androgen receptor expression. Proxalutamide improves lung opacities in hospitalized COVID-19 patients when compared to placebo. See clinical trial NCT04728802.

Read origin protocol (ROP) has the biomedical art-recognized meaning of a computational protocol that aims to discover the source of all reads, including those originating from repeat sequences, recombinant B and T cell receptors, and microbial communities. The Read Origin Protocol was developed to determine what the unmapped reads represented. Mangul al., Genome Biology 19, 36 (2018).

Sabizabulin (VERU-111, IUPAC name [2-(1H-Indol-3-yl)-1H-imidazol-5-yl]-(3,4,5-trimethoxyphenyl)methanone, CAS Number 1332881-26-1) is an antiandrogen medication, that blocks the translocation of the androgen receptor to the nucleus. Sabizabulin (9-mg) capsules and matching placebo capsules are manufactured and tested by CoreRx (Clearwater, FL, USA). Sabizabulin is being studied as a mitotic inhibitor and chemotherapeutic agent in castration-resistant metastatic prostate cancer and in SARS-CoV-2 (COVID-19) infections.

Sebum has the biomedical art-recognized meaning. The sebaceous gland has several biological functions in hormonal and immune modulation. The number of sebaceous glands remains much the same throughout life, while its activity increases, driven by elevated androgen levels during puberty. As an androgen target tissue, the sebaceous glands possess steroidogenic enzymes that convert adrenal and ovarian precursor androgens into active androgens. Differentiated sebocytes have the highest androgen receptor density. The sebum hypothesis proposed that disruption of the pilosebaceous structure initiates the chain, and the leakage of sebum triggers T-lymphocyte recognition and proliferation, leading to a delayed-type hypersensitivity reaction in sensitive individuals. See Yang et al., Chinese Journal of Plastic and Reconstructive Surgery, 4, 44-48 (2022).

Sepsis has the biomedical art-defined meaning of a life-threatening condition that arises when the body's response to infection injures its tissues and organs. Bone et al., Chest, 101, 1644-1655 (1992); Singer et al., JAMA, 315, 801-810 (February 2016). Sepsis is a life-threatening organ dysfunction due to a dysregulated host response to infection. Despite declining age-standardized incidence and mortality, sepsis remains a significant cause of health loss worldwide. Rudd et al., The Lancet, 395(10219), 200-211 (Jan. 18, 2020). A timely implementation of targeted interventions improves sepsis outcomes.

STAR aligner is the Spliced Transcripts Alignment to a Reference (STAR), a fast RNA-seq read mapper, with support for splice-junction and fusion read detection. STAR aligns reads by finding the Maximal Mappable Prefix (MMP) hits between reads (or read pairs) and the genome, using a Suffix Array index. Different parts of a read can be mapped to different genomic positions, corresponding to splicing or RNA-fusions. The genome index includes known splice-junctions from annotated gene models, allowing for sensitive detection of spliced reads. STAR performs local alignment, automatically soft clipping ends of reads with high mismatches. Dobin et al., STAR: Ultrafast universal RNA-seq aligner. Bioinformatics, 29(1), 15-21 (January 2013).

Subject and patient have the biomedical art-recognized meanings. The term patient includes human and other mammalian subjects that receive either prophylactic or therapeutic treatment.

Systemic treatment has the biomedical art-recognized meaning. Transmembrane protease serine 2 (TMPRSS2) has the biomedical art-recognized meaning. Traumatic lung injury has the biomedical art-recognized meaning. Treatment for acute respiratory distress syndrome (ARDS) has the biomedical-art recognized meaning. Corticosteroids can be therapeutic. See Prescott & Rice, Corticosteroids in COVID-19 ARDS: Evidence and hope during the pandemic. JAMA, 324, 1292-1295 (2020). Other treatments are known by persons having ordinary skill in the medical art.

Treatment for COVID-19 has the biomedical-art recognized meaning. Corticosteroids can be therapeutic. See Prescott & Rice, Corticosteroids in COVID-19 ARDS: Evidence and hope during the pandemic. JAMA, 324, 1292-1295 (2020). Other treatments are known by persons having ordinary skill in the medical art. See Waterer & Rello, Infectious Diseases and Therapy (2020). See also Beigel et al., Remdesivir for the treatment of Covid-19—Preliminary Report. New England Journal of Medicine (2020). Several agents were explored in clinical trials as treatments for COVID-19. The most promising of these treatments are remdesivir, a viral RNA-polymerase inhibitor, remdesivir plus the Janus kinase (JAK) inhibitor, baricitinib, and dexamethasone, a glucocorticoid without mineralocorticoid effect. Beigel et al., New England Journal of Medicine, 383(19): 1813-1826 (2020); Kalil et al., New England Journal of Medicine, 384(9): 795-807 (2021); Dexamethasone in Hospitalized Patients with Covid-19—Preliminary Report. New England Journal of Medicine, 384(8) (2020). Remdesivir reduce the median time to recovery of hospitalized COVID-19 patients to ten days, down from fifteen days, as observed with placebo. In addition to the therapies disclosed in this specification, therapies efficacious for treating COVID-19 patients are provided by the United States Center for Disease Control. Among the therapies efficacious for treating COVID-19 patients are nirmatrelvir with ritonavi (Paxlovid®), remdesivir (Veklury®), bebtelovimab, and molnupiravir (Lagevrio®). Regarding the standard of care for the treatment of COVID-19, there is a perceived lack of efficacy in mortality for the oral medication Paxlovid. New therapies are in the process of being discovered and recommended.

Treatment for sepsis has the biomedical-art recognized meaning. Sepsis is treatable, and timely implementation of targeted interventions improves outcomes. The Mayo Clinic informs the public that several medications are used in treating sepsis and septic shock. They include antibiotics. Broad-spectrum antibiotics, which are effective against a variety of bacteria, are usually used first. After learning the results of blood tests, a doctor may switch to a different antibiotic that's targeted to fight the specific bacteria causing the infection. They include intravenous fluids and vasopressors. Other medications include low doses of corticosteroids, insulin to help maintain stable blood sugar levels, drugs that modify the immune system responses, and painkillers or sedatives.

This specification does not concern a process for cloning humans, methods for modifying the germ line genetic identity of humans, uses of human embryos for industrial or commercial purposes, or procedures for modifying the genetic identity of animals likely to cause them suffering with no substantial medical benefit to humans or animals resulting from such processes.

It is important to note that any therapeutic agent disclosed herein can be used in combination with a corticosteroid. For example, sabizabulin can be administered with any other therapeutic agents (e.g., combo therapies such as one or more corticosteroids. In an example, when a subject is exposed to an anti-androgen, the body increases testosterone, so there is a need to add corticosteroid with it. Dexamethasone can decrease effects of male hormones. In addition to the administration of anti-androgen small molecules, the method may also involve the use of corticosteroids in combination, before and/or after, with these molecules. The combination of anti-androgen small molecules with corticosteroids can potentially enhance the therapeutic effects and provide additional benefits in treating the condition and/or sepsis. Corticosteroids are known for their anti-inflammatory and immunosuppressive properties, which can be advantageous in managing inflammatory conditions and sepsis.

A wide range of corticosteroids can be administered along with sabizabulin or other anti-androgen small molecules. These corticosteroids include, but are not limited to, dexamethasone, prednisone, prednisolone, methylprednisolone, hydrocortisone, betamethasone, triamcinolone, fluticasone, budesonide, mometasone, beclomethasone, ciclesonide, flunisolide, and cortisone. The selection of a specific corticosteroid may depend on the individual subject's condition, response to treatment, and any potential contraindications.

The combination therapy involving anti-androgen small molecules and corticosteroids may be tailored to the specific needs of the subject. The dosage and administration schedule of both the anti-androgen small molecules and corticosteroids can be adjusted based on the severity and stage of the condition and/or sepsis, as well as the subject's response to treatment. This approach allows for a more personalized treatment regimen that aims to optimize therapeutic outcomes.

The technology herein can be utilized to treat netosis, neutrophil inflammation, (e.g., puss, acne), cytokine storm, and/or COVID-19.

Administration of sabizabulin. In one example, treatment with sabizabulin 9 mg once daily, an oral, first-in-class, chemical entity, microtubule disruptor that has dual anti-inflammatory and antiviral properties.

Several other existing, approved medications could be repurposed for COVID-19. The mineralocorticoid receptor antagonist spironolactone is a known androgen receptor antagonist. Dhurat et al., Dermatol. Ther., 34(1) (2021). 5-alpha reductase inhibitors such as dutasteride and finasteride are prescribed for hyperandrogenic features involving dihydrotestosterone activity, such as labeled and off-label use in dermatology to treat androgenetic alopecia and the labeled indication use in urology for benign prostatic hyperplasia. Both spironolactone and dutasteride decrease the expression of TMPRSS2 and angiotensin converting enzyme 2 (ACE2) in men, making them candidates for generic, low-cost COVID-19 therapy. Samuel et al., Cell Stem Cell, 27(6), 876-889.e12 (2020).

Dexamethasone benefits the most severe COVID-19 patients. Among those patients requiring mechanical ventilation, dexamethasone was shown to reduce mortality compared to placebo. Dexamethasone also has known to have mild antiandrogen effects, by reducing the expression of adrenal corticosteroids.

Baseline COVID-19 8-pjoint ordinal scale. The ordinal scale is defined as follows: 8. Death; 7. Hospitalized, on invasive mechanical ventilation or ECMO; 6. Hospitalized, on non-invasive ventilation or high flow oxygen devices; 5. Hospitalized, requiring supplemental oxygen; 4. Hospitalized, not requiring supplemental oxygen-requiring ongoing medical care (COVID-19 related or otherwise; 3. Hospitalized, not requiring supplemental oxygen—no longer requires ongoing medical care; 2. Not hospitalized, limitation on activities; 1. Not hospitalized, no limitations on activities. Mortality is the most objective outcome of interest in COVID-19.

The inventors previously proposed using RNA sequencing data from critically ill patients to identify antibodies for other diseases. Typically, only RNA sequencing data that aligns to the organism of interest is analyzed. However, some groups recently began analyzing data that is unmapped to an organism of interest. Mangul et al., Genome Biology, 19, 36 (2018). The inventors previously showed many uses for unmapped data. Monaghan et al., medRxiv (2021).

The Surviving Sepsis Campaign standardized treatment for sepsis that includes blood cultures before broad-spectrum antibiotics and initiation of antibiotics within one hour. See Evans et al. (2021). In a multivariate analysis of factors impacting mortality in patients with septic shock, the time to begin antibiotic treatment was the most impactful variable. Kumar et al. showed this impact, reporting a 79.9% survival in septic shock patients with antibiotics in the first hour and a reduction of 7.6% for every hour delay. Kumar et al., (2006). Vazquez-Guillamet et al. determined that the number needed to treat with antibiotics to save one life was five. Vazquez-Guillamet et al., (2014). Faster pathogen identification improves sepsis outcomes by guiding antibiotic selection. Current methods take too much time, such as days. Sepsis kills in hours.

RNA sequencing methods. The analytical test to assess whether the subject or patient is an appropriate candidate for treatment by the administration of sabizabulin or a therapeutically similar antiandrogen can be by RNA sequencing to provide a diagnostic for sepsis that can also monitor the indicia of treatment and recovery (bacterial counts reduce, physiology returns to steady-state). See Intl. Pat Publ. WO 2021/163692 and U.S. Pat. Publ. US 2022/0340972. This technology can be used for many other hospital conditions, particularly those needing an intensive care unit stay with the attendant risk of bacterial infection, such as trauma, stroke, myocardial infarction, or major surgery. This technology provides an initial diagnostic for sepsis that can also monitor the indicia of treatment and recovery (bacterial counts reduce, physiology returns to steady-state). The first step is for one of ordinary skill in the molecular biological art to obtain RNA sequencing from a body sample. The second step is for one to align the RNA sequencing data (reads) to the genome of interest. The third step is to select the un-mapped reads and analyze the reads using a Read Origin Protocol (ROP). One selects the mapped reads and then uses a program that enables detection and quantification of alternative RNA splicing events to identity gene expression, RNA splicing events, alternative transcription start/end, or RNA splicing entropy. The next step is to identify bacteria present in the sample. From the ROP, one of ordinary skill in the molecular biological art identifies bacteria present in the sample. The next step is to identify the viruses present in the sample. From the ROP, the next step is to identify the T/B cell epitopes present in the samples. In yet another embodiment, one uses the T/B cell epitopes identified with PCA to identify likely sepsis samples. RNA sequencing data be used in several ways. (1) Identification of biomarkers. Rather than need to pick a subset to test for, RNA sequencing data can identify genes with increased expression that would correlate to biomarkers of interest. (2) Identification of biomarkers. RNA sequencing data allows for analysis of processes such as RNA splicing. The method of RNA splicing entropy can be quantified and grouped according to a Principal Component Analysis into sick or not sick. RNA lariats can also be identified in sequencing data and used as a potential biomarker. All biomarkers can be followed over time to assess for resolution of the sepsis. (3) Use of un-mapped reads in sepsis. RNA sequencing typically aligns with the genome of reference (i.e., the human genome). Reads that are not aligned to the human genome are discarded (the percentage of un-mapped reads could itself be a biomarker). These un-mapped reads could be of two major potential interests. (4) Identification of the microbe causing the infection. The unmapped reads can be referenced to the genome of disease-causing microbes (bacteria, viruses, fungi, etc.) to identify the causative organism and start treatment earlier. Serial measurements can also assess the effectiveness of treatment.

Additional information on an analytical test to assess the progress or success of the administration of sabizabulin or therapeutically similar antiandrogen: Other RNA sequencing methods. The analytical test to assess the progress or success of the administration of sabizabulin or therapeutically similar antiandrogen can be an RNA sequencing test to assess lung improvement in patients with severe COVID-19. See Intl. Pat. Publ. WO 2023/086827. Therapeutic antibodies of certain types of antibodies to COVID-19 are identified early by targeted diagnostic testing can be given to other COVID-19 patients to enhance their recovery.

The analytical test to assess the progress or success of the administration of sabizabulin or therapeutically similar antiandrogen can be supplemented by other tests used to assess androgen receptor inhibition to treat sepsis and shock. See Intl. Pat. Publ. WO 2022/225960.

Radiological scans. The radiological changes follow the improvement in clinical outcomes with proxalutamide. X-ray scans are informative lung assessments. CT scans are more informative than X-ray scans.

RNA extraction, sequencing, data protection and quality assurance. Blood is collected directly from the patient into PAXgene tubes (Qiagen, Germantown, MD, USA) to stabilize the RNA. The tubes were then stored as described by the manufacturer. All samples require at least 1400 nanograms of RNA for deep sequencing. With the PAXgene system, one routinely obtains >3000 nanograms. After RNA samples are processed, they are sent out for RNA sequencing. Because of the high concentration of globin and ribosomal RNA in blood samples, these samples are then further processed at the sequencing company to reduce globin RNA and human ribosomal RNA. This optimizes the yield of clinically relevant reads. Each sample are sent out for deep RNA sequencing with a goal of 100 million reads per sample.

Assessment of clinical information. RNA sequencing data are interpreted with clinical data collected from the electronic medical record including endpoints such as mortality, Intensive Care Unit length of stay, hospital length of stay, SOFA score (Shankar-Hari et al. (2016)), ventilator days, renal failure, ARDS (Ferguson et al. (2012)). Culture data are based upon the test results in the microbiology lab and are the gold standard. Clinical response to antibiotics are also be tracked to see if the treatment based upon microbiology data is correct. Changes in treatment are assessed to ensure culture data is utilized in treatment and antimicrobial stewardship practices are being followed.

Computing resources. Computational biology work is performed on servers on premise. These servers are secured because they contain clinical data. All HIPAA standards are applied. The server operates on 6× VxRail E560F nodes (PowerEdge R640 1 U rack mount servers) and has dual Intel Xeon Platinum 8260 (24c) 2.4 Ghz with 1,152 GB RAM, 2×1.6 TB SAS SSD cache, 8×7.68 TB SAS SSD capacity, 4×10 Gb data ports, and 1×1 Gb iDRAC management port. This server includes vSphere Enterprise Plus with 3 Years 24×7 Mission Critical Support per node configured to provide the computational infrastructure. The server consists of 288c (691.2 GHz) CPU, and 6.75 TB RAM. Storage estimates reflect 368.64 TB RAW/222 TB usable memory on a RAID6 configuration with 20% vSAN overhead. This server manages all large data sets from RNA sequencing. Because of the depth of sequencing that is needed for RNA splicing analysis (100 million reads vs. 40 million), more data is generated from both sequencing and analysis.

Polymerase chain reaction (PCR) design. Optimized PCR parameters are essential to ensuring accuracy and reproducibility in qPCR reactions. See Bustin & Huggett (2017); Bustin, Mueller, & Nolan (2020)). Target selection is critical. The preliminary data demonstrate that bacterial reads are measurable from patient with bacteremia and pneumonia and that the reads can be aligned to the organism's genome. RNA sequencing data accumulated from patients with bacteremia or pneumonia due to the specified pathogens are used to identify sequences of interest. These sequences are compared to a pan genome of the same organism to confirm the target is generalizable to the pathogen. Wang et al. (2022). The inventors use Beacon Designer (Premier Biosoft) to design several primer/probe combinations for the sequences, the specificity of which are confirmed by BLAST searches. Primers with low specificity, dimer formation, or that create amplicons with complex secondary structures are excluded. Bustin & Huggett (2017). Primer-BLAST (NCBI) are used as an independent, complementary design strategy; primers identified by both approaches are prioritized. PCR reactions are optimized in the laboratory for temperature and primer concentration for the mastermix. The objective is to establish a standard set of testing conditions.

Testing the PCR. The PCR tests are validated in two ways. First, cDNA libraries used for RNA sequencing are tested. Next, RNA from the blood of patients, both with and without the infection, are used as templates for cDNA synthesis and then PCR. PCRs are applied to the samples from RNA sequencing and an independent cohort of patients to validate the assays. Several primer combinations are evaluated for each target sequence. Bustin & Huggett (2017). SYBR green methodology are used initially to prioritize different primer combinations. Hydrolysis (Taqman) probes for qPCR, which were already designed with the primers, is then synthesized for the prioritized primer combinations.

Rigor and reproducibility. RNA is less stable than DNA because of its vulnerability to hydrolysis. This invention uses RNA sequencing and uses methodology to ensure stability. The preliminary data show isolated RNA from critically ill patients and high quality RNA sequencing results. The inventors also focus on isolation methods that are standard and can easily be applied followed so the results can be translated to clinical practice. To enhance robustness during development, it is standard practice for each step of the PCRs (setup, cycling, analysis) to be performed in separate rooms, reducing reactions being contaminated with amplicons from prior runs.

Biological variables. Variables such as age (patients are included across the lifespan, weight, and medical co-morbidities are collected and compared across groups. If these variables, or sex, are significantly different (t test or rank sum), these factors are adjusted for in the analysis via regression.

Identification of antibodies. Alignment files are parsed for reads mapping to the V(D)J locus using ImReP9 to identify novel antibodies produced by the patients with active COVID-19 disease requiring intensive care unit admission. The resulting CDR3 sequences were then compared across survivors and non-survivors. Only sequences that appeared in every patient in each group were considered distinct to that group. Comparison is done using a NCBI Blast program, with a threshold of 66% length match and 70% sequence match. Querying sequences by time point, across time points, further filtered blast output and survivors vs. non-survivors by time point and across time points.

Statistical analysis. SigmaPlot 14.5 (Sysstat Software) is used for analysis. T-tests are used to compare survivors and non-survivors, but paired t-tests are used to compare across time points. Alpha is set at 0.05.

Other measurements. Viral clearance is measured by repeat PCR testing was an outcome measure in multiple studies.

Lung improvement can be measured by X-ray scans or by CT scans. CT scans are more informative than X-ray scans. An assay to measure C-reactive protein is usually enough to detect inflammation. Testosterone-binding receptor protein analyses can be performed on patient samples.

Test for coronavirus clearance. In the biomedical art, the progress of COVID-19 treatment is measured by nasopharyngeal viral clearance. See Lin et al., Accelerated viral clearance and symptom resolution in symptomatic COVID-19 outpatients treated with antiandrogens. Viral clearance determined by repeat PCR testing is used as an outcome measure in multiple studies.

### EXAMPLES

### Example 1. Testing of Pharmaceutically Acceptable Salts, Hydrates, and Solvates

In this prophetic example, sabizabulin is obtained at about the five-gram scale, with a purity above 99% (assay) and production of various pharmaceutically acceptable salts is initiated to improve clinical attributes such as solubility and stability.

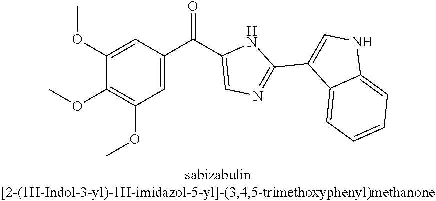 [SMILES: COc1cc(C(=O)c2cnc(-c3c[nH]c4ccccc34)[nH]2)cc(OC)c1OC]

Various pharmaceutically acceptable anions are associated after a charge is induced, but IR investigations (and NMR) are initiated to determine exact position of anion association, while proposed positions are shown below. In some embodiments, the—Anion shown below includes bisulfate, chloride, bromide, carbide, fluoride, hydride, iodide, nitride, phosphide, oxide, sulfide, selenide, azide, peroxide, triodide, carbonate, chlorate, chromate, dichromate, dihydrogen phosphate, hydrogen carbonate, hydrogen sulfate, hydrogen sulfite, hydroxide, hypochlorite, mono-hydrogen phosphate, nitrate, nitrite, perchlorate, permanganate, peroxide, phosphate, sulfate, sulfite, superoxide, thiosulfate, silicate, metasilicate, aluminum silicate, acetate, formate, oxalate, or a combination thereof.

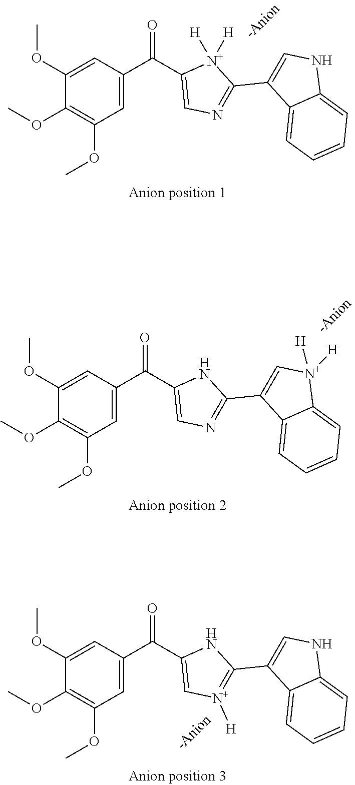 [SMILES: COc1cc(C(=O)C2=CN=C(c3c[nH]c4ccccc34)[NH2+]2)cc(OC)c1OC ; COc1cc(C(=O)c2cnc(C3=C[NH2+]c4ccccc43)[nH]2)cc(OC)c1OC ; COc1cc(C(=O)c2c[nH+]c(-c3c[nH]c4ccccc34)[nH]2)cc(OC)c1OC]

During these investigations, formation of hydrates and solvates is also investigated by acquiring ATR-IR spectra of the solids, after evaporating solvents and/or water, and observing significant increases in hydrogen bonding bands. As discussed above, the present disclosure includes the use of pharmaceutically acceptable salts of compounds of the invention in the compositions and methods of the present invention. In certain embodiments, contemplated salts of the invention include, but are not limited to; bisulfate; chloride; alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I-tartaric acid, thiocyanic acid, p-toluenesulfonic acid, trifluoroacetic acid, and undecylenic acid salts. In some embodiments, an N—H in sabizabulin can become charged and a corresponding salt formed at or near the charge.

The invention contemplates that the above-described compounds or combinations can be derivatized or can be structurally altered, for example, by addition or substitution of one or more atoms using a radioisotope or using a different element (e.g., B or boron in place of C or carbon), by removal of an ester or by addition of a salt form, an amino acid, a sugar, or a peptide. Hydrates and/or solvates can be formed by 1) dissolving the molecule in water and/or solvent and slowly drying, whereby water and/or solvent remain hydrogen bonded with OH groups in the molecule or associated with the molecule. A formation of a hydrate or solvate can typically be confirmed by an attenuated total reflection (ATR) infrared spectrum acquired from the solid-state sample. The ATR spectrum of a hydrate or solvate will typically show increased broad bands (indicating hydrogen bonding) above about 3200 cm¹, as compared to the non-hydrate or non-solvate solid sample. In some embodiments, the above-described compounds are attached to or associated with a targeting moiety. In some embodiments, the targeting moiety is a particle or an antibody with affinity for a specific type of cell. While various crystal structure are contemplated, these investigations will also be initiated.

### Example 2. Prophetic Testing of Methods

Sabuzabulin was studied as capsules (e.g., drug in capsule) and a feeding tube was used in some cases. Xtandi (enzalutamide) was also capsules. Proxalutamide was available in tablets, and we had experience of crushing the tablets and injecting into the nasal-gastric feeling tube with success in a suspension.

In this prophetic example hereafter, according to some aspects, the techniques described herein relate to a method for treating a dermatological condition and/or a dermatological manifestation of sepsis in a subject in need thereof, the method including the steps of: (1) executing a dermatological analytical test on the subject, whereby the test will assess whether or not the subject is an appropriate candidate for a treatment by an administration of a therapeutically effective amount of an anti-androgen small molecule or a potential anti-androgen small molecule; (2) if the subject is an appropriate candidate after the test in step (1), administer the therapeutically effective amount of the anti-androgen small molecule and/or the potential anti-androgen small molecule to the subject; whereby the dermatological condition and/or the dermatological manifestation of sepsis in the subject is improved compared to the same subject without the administering of the therapeutically effective amount of the anti-androgen small molecule and/or the potential anti-androgen small molecule to the subject.

In some embodiments, the techniques described herein relate to a method, further including the step of: (3) executing a dermatological analytical test to assess success of the administration of the anti-androgen small molecule or a potential anti-androgen small molecule, wherein the dermatological analytical test includes a skin biopsy, a skin swab culture, a skin imaging test, a skin lesion assessment, a skin biomarker test, a skin microbiome analysis, or a combination thereof.

According to some aspects, the techniques described herein relate to a method, wherein the small molecule includes sabizabulin or a pharmaceutically acceptable hydrate, solvate, and/or salt form of sabizabulin, and wherein sabizabulin or its pharmaceutically acceptable form is administered topically, orally, or by injection to treat the dermatological condition and/or the dermatological manifestation of sepsis.

In some embodiments, the techniques described herein relate to a method, wherein the anti-androgen small molecule or the potential anti-androgen small molecule is administered in a therapeutically effective amount that is determined based on the severity and stage of the dermatological condition and/or the dermatological manifestation of sepsis in the subject, and wherein the severity and stage are assessed using a dermatological grading scale or a dermatological scoring system.

According to some aspects, the techniques described herein relate to a method, wherein the improvement in the dermatological condition and/or the dermatological manifestation of sepsis is compared to the same subject without the administering of the therapeutically effective amount of the anti-androgen small molecule or the potential anti-androgen small molecule, and wherein the improvement is measured by one or more dermatological parameters, such as skin redness, skin scaling, skin thickness, skin itching, skin pain, skin lesion size, skin lesion number, or skin wound healing rate.

In some embodiments, the techniques described herein relate to a method, wherein the anti-androgen small molecule or the potential anti-androgen small molecule is at least one of an anti-androgen small molecule or a potential anti-androgen small molecule that acts on the androgen receptor pathway in the skin, and wherein the androgen receptor pathway is involved in the pathogenesis of the dermatological condition and/or the dermatological manifestation of sepsis.

According to some aspects, the techniques described herein relate to a method, wherein the dermatological analytical test substantially assesses the appropriateness of the subject for the treatment by measuring one or more dermatological biomarkers, dermatological clinical symptoms, or dermatological risk factors associated with the dermatological condition and/or the dermatological manifestation of sepsis, and wherein the dermatological biomarkers include skin cytokines, skin chemokines, skin hormones, skin growth factors, skin antimicrobial peptides, or skin matrix metalloproteinases.

In some embodiments, the techniques described herein relate to a method, wherein the administering is substantially of the therapeutically effective amount of the anti-androgen small molecule or the potential anti-androgen small molecule, and wherein the therapeutically effective amount is sufficient to improve the dermatological condition and/or the dermatological manifestation of sepsis in the subject, as evidenced by a reduction in skin inflammation, skin infection, skin wound, skin lesion, skin scar, skin itch, skin pain, skin scaling, skin thickening, skin redness, or skin pigmentation.

According to some aspects, the techniques described herein relate to a method, wherein the dermatological condition includes acne, rosacea, psoriasis, atopic dermatitis, seborrheic dermatitis, contact dermatitis, lichen planus, hidradenitis suppurativa, pemphigus, bullous pemphigoid, vitiligo, alopecia areata, skin cancer, skin infection, skin wound, or any skin sign, symptom or condition that mimics a dermatological manifestation of sepsis.

In some embodiments, the techniques described herein relate to a method, wherein the dermatological condition and/or the dermatological manifestation of sepsis is caused by one or more of a bacterial skin infection, a viral skin infection, a fungal skin infection, a parasitic skin infestation, an inflammatory skin condition, or a systemic inflammatory response syndrome that affects the skin.

According to some aspects, the techniques described herein relate to a method, wherein the subject is a human patient diagnosed with or at risk of developing the dermatological condition and/or the dermatological manifestation of sepsis, and wherein the subject has a medical history, a family history, a genetic predisposition, or a lifestyle risk factor for the dermatological condition and/or the dermatological manifestation of sepsis.

In some embodiments, the techniques described herein relate to a method, wherein the dermatological analytical test is performed prior to the administering and is used to select subjects who are likely to respond to the treatment, and wherein the dermatological analytical test is a genetic test, a blood test, a urine test, a stool test, a skin test, or a combination thereof.

According to some aspects, the techniques described herein relate to a method, wherein the administering is performed after the assessing and only in subjects who are determined to be appropriate candidates for the treatment based on the results of the dermatological analytical test, and wherein the appropriate candidates have a dermatological biomarker level, a dermatological clinical symptom score, or a dermatological risk factor profile that meets a predetermined threshold or criterion.

In some embodiments, the techniques described herein relate to a method, wherein the improvement is assessed after the administering at one or more time points to monitor the effectiveness of the treatment, and wherein the time points are selected based on the pharmacokinetics, the pharmacodynamics, or the therapeutic window of the anti-androgen small molecule or the potential anti-androgen small molecule in the skin.

According to some aspects, the techniques described herein relate to a method, wherein the anti-androgen small molecule or the potential anti-androgen small molecule is administered topically, orally, intravenously, intramuscularly, or subcutaneously using a suitable pharmaceutical formulation, and wherein the pharmaceutical formulation is a cream, a lotion, an ointment, a gel, a spray, a foam, a solution, a suspension, an emulsion, a microemulsion, a nanoemulsion, a liposome, a niosome, a nanoparticle, a microparticle, a microneedle, a patch, or a combination thereof.

In some embodiments, the techniques described herein relate to a method, wherein the anti-androgen small molecule or the potential anti-androgen small molecule is administered in a single dose or in multiple doses according to a therapeutic regimen that is tailored to the individual subject, and wherein the therapeutic regimen is adjusted based on the dermatological response, the dermatological side effect, or the dermatological tolerance of the subject to the treatment.

According to some aspects, the techniques described herein relate to a method, wherein the therapeutically effective amount is determined based on the severity of the dermatological condition and/or the dermatological manifestation of sepsis in the subject, as well as other dermatological factors such as the skin type, the skin color, the skin thickness, the skin barrier function, the skin microbiome composition, or the skin pharmacokinetics of the subject.

In some embodiments, the techniques described herein relate to a method, wherein the anti-androgen small molecule or the potential anti-androgen small molecule is selected based on the results of the dermatological analytical test, and wherein different anti-androgen small molecules or potential anti-androgen small molecules may be selected for different subjects depending on their individual dermatological test results, such as their skin androgen receptor expression level, their skin androgen sensitivity, their skin androgen metabolism, or their skin androgen-related genetic polymorphisms.

According to some aspects, the techniques described herein relate to a method, wherein the dermatological analytical test assesses whether or not the subject is an appropriate candidate for the treatment by evaluating one or more dermatological genetic, dermatological biochemical, or dermatological clinical parameters that are predictive of the subject's dermatological response to the treatment, and wherein the dermatological genetic parameters include skin androgen receptor gene mutations, skin androgen-metabolizing enzyme gene polymorphisms, or skin androgen-regulated gene expression profiles.

In some embodiments, the techniques described herein relate to a method, wherein the therapeutically effective amount is administered to the subject assessed as an appropriate candidate for the treatment, and wherein the subject is monitored for dermatological adverse reactions or dermatological side effects during the course of the treatment, such as skin irritation, skin dryness, skin flaking, skin peeling, skin redness, skin itching, skin burning, skin stinging, skin pain, skin swelling, skin blistering, skin crusting, skin pigmentation, skin photosensitivity, skin atrophy, skin striae, skin telangiectasia, skin hypertrichosis, or skin acneiform eruptions.

### Other Embodiments

Specific compositions and methods of androgen receptor inhibition to treat sepsis and shock were described. The scope of the invention should be defined solely by the claims. A person having ordinary skill in the art will interpret all claim terms in the broadest possible manner consistent with the context and the spirit of the disclosure.

The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as those skilled in the biomedical art recognize.

All terms should be interpreted in the broadest possible manner consistent with the context when interpreting the disclosure. Unless otherwise defined, all technical and scientific terms used in this specification have the same meaning as commonly understood by persons having ordinary skill in the biomedical art. This invention is not limited to the particular methods, protocols, and reagents described in this specification and can vary in practice. The invention is defined only by the claims.

When a range of values is provided, each intervening value, to the tenth of the unit of the lower limit, unless the context dictates otherwise, between the upper and lower limit of that range and any other stated or intervening value in that range of values.

### BACKGROUND REFERENCES

A person having ordinary skill in a medical art with access to a library can use these patents, patent applications, and scientific references as guidance to predictable results when making and using the invention.

Patent literature: US 2019/0055192 (University of Tennessee Research Foundation), Selective androgen receptor modulator and methods of use thereof discloses a method for applying diarylhydantoin compounds and methods of use of a selective androgen receptor modulators and for the treatment of the indicated diseases, disorders or conditions. The SARM compound of this invention is administered in with an agent treating the endocrine system, such as a steroidal or nonsteroidal androgen receptor antagonist. The patent application does not explicitly disclose proxalutamide.
US 2020/0155521 (Arqule, Inc.), Pharmaceutical combination for treatment of cancer. This patent application teaches the use of proxalutamide to treat sepsis or the treatment or prevention of a cell proliferative disorder in a subject in need thereof.
US 2022/0340972 (Monaghan et al.), RNA sequencing to diagnose sepsis and other diseases and conditions.
WO 2020/198062 (Achillion Pharmaceuticals, Inc.), Pharmaceutical compounds for the treatment of complement mediated disorders.
WO 2021/108637 (Cedars-Sinai Medical Center), Compositions and methods for treating diseases and conditions by depletion of mitochondrial or genomic DNA from circulation.
WO 2021/154687 (Gilead Sciences, Inc.), Methods for treating SARS COV-2 infections. See also US 2023/0346812 (Chilar et al.).
WO 2021/163692 (Rhode Island Hospital), RNA sequencing to diagnose sepsis.
WO 2021/163692 (Rhode Island Hospital), RNA sequencing to diagnose sepsis, published Aug. 19, 2021. Deep RNA sequencing is a technology that provides an initial diagnostic for sepsis that can also monitor the indicia of treatment and recovery (bacterial counts reduce, physiology returns to steady-state).
WO 2022/225960 (Rhode Island Hospital), Androgen receptor inhibition to treat sepsis and shock.
WO 2023/086827 (Rhode Island Hospital), Predicting COVID-19 antibodies among survivors with deep RNA sequencing. Therapeutic antibodies of certain types of antibodies to COVID-19 are identified early by targeted diagnostic testing can be given to other COVID-19 patients to enhance their recovery.

### Non-Patent Literature

Barnette, et al., Oral sabizabulin for high-risk, hospitalized adults with Covid-19: Interim analysis. NEJM Evid., 1 (9) (Jul. 6, 2022). Sabizabulin treatment resulted in about a 25% absolute reduction in deaths compared with placebo in hospitalized patients with moderate to severe Covid-19 at high risk for ARDS and death, with a lower incidence of adverse and serious adverse events compared with placebo. (Funded by Veru, Inc.; ClinicalTrials.gov number, NCT04842747.) This study was funded by Veru, Inc.
Based on Science, What is the connection between COVID-19 and sepsis? National Academies (Feb. 14, 2022).
Beigel et al., Remdesivir for the treatment of Covid-19—Final report. New England Journal of Medicine, 383(19), 1813-1826 (2020).

Bhowmick et al., COVID-19 and androgen-targeted therapy for prostate cancer patients, Endocrine-Related Cancer, vol. 27, no. 9, 5 Jun. 2020 (2020-06-05), pages R281-R292, XP055950411. Bhowmick discloses a method for applying diarylhydantoin compounds targeting TMPRSS2 and/or ACE2 through androgen signaling regulation for COVID-19 treatment, treatment with estradiol (prolonged) or enzalutamide significantly downregulated TMPRSS2, androgen regulation ofTMPRSS2 and ACE2 as a means to inhibit SARS-CoV-2 viral entry and thus infection.
Bussani et al. Persistence of viral RNA, pneumocyte syncytia and thrombosis are hallmarks of advanced COVID-19 pathology. EbioMedicine, 61, 103104 (2020).
Cadegiani et al. Final results of a randomized, placebo-controlled, two-arm, parallel clinical trial of proxalutamide for hospitalized COVID-19 patients. A multiregional, joint analysis of the Proxa-Rescue AndroCoV trial. Cureus, 13, e20691 (2021).
Cadegiani et al., Clinical symptoms of hyperandrogenic women diagnosed with COVID-19. J. Eur. Acad. Dermatol. Venereol., 35(2) (2021).
Cadegiani et al., Early antiandrogen therapy with dutasteride reduces viral shedding, inflammatory responses, and time-to-remission in males with COVID-19: A Randomized, double-blind, placebo-controlled interventional trial (EAT-DUTA AndroCoV Trial—Biochemical). Cureus (February 2021).
Cadegiani et al., Efficacy of proxalutamide in hospitalized COVID-19 patients: A randomized, double-blind, placebo-controlled, parallel-design clinical trial. medRxiv, 2021.06.22.21259318. Check the computed tomography (CT) scan results of the clinical trial.
Cadegiani et al., Potential risk for developing severe COVID-19 disease among anabolic steroid users. BMJ Case Reports, 14(2), e241572 (2021). A young bodybuilder that was developing severe COVID-19 symptoms while in use of the dihydrotestosterone analogue oxandrolone as an anabolic steroid, who had striking symptom resolution and laboratory improvement within twenty-four hours of starting proxalutamide.
Cadegiani et al., Proxalutamide improves lung injury in hospitalized COVID-19 patients—An analysis of the radiological findings of the Proxa-Rescue AndroCoV Trial. medrxiv.org.
Cadegiani et al., Proxalutamide significantly accelerates viral clearance and reduces time to clinical remission in patients with mild to moderate COVID-19: results from a randomized, double blinded, placebo-controlled trial, Cureus, Vol. 13, No. 2, pages 1-8 (Feb. 22, 2021). XP055966260.

Cadegiani, Lin, Goren, & Wambier, Potential riskfor developing severe COVID-19 disease among anabolic steroid users. BMJ Case Rep., 14(2), e241572 (2021). A severe case of COVID-19 was observed in an otherwise healthy 28-year-old man who had taken oxandrolone as an anabolic steroid. As part of an experimental antiandrogen treatment for hyperandrogenic men suffering from COVID-19, he was administered a single of the antiandrogen proxalutamide. This case report was retracted because the evidence of informed consent was inadequate. See BMJ Case Report, 16(11), e241572ret (November 92023).
Cadegiani, Pre-RCT AndroCoV Trial—COVID-19 public dataset 2020-10-31. OSF. Published 2020.

Cheema et al., Antiandrogens for the treatment of COVID-19 patients: A meta-analysis of randomized controlled trials. Journal of Medical Virology, Volume 95, Issue 4, e28740. Antiandrogens may carry a potential benefit as a therapeutic agent against COVID-19. The authors performed a quantitative synthesis of data to quantify the benefits of antiandrogens. The authors systematically searched PubMed/MEDLINE, Cochrane Library, clinical trial registers, and reference lists of included studies to identify relevant randomized controlled trials (RCTs). They found that antiandrogens yielded a significant mortality benefit, but on subgroup analysis, only proxalutamide/enzalutamide and sabizabulin were found to significantly reduce mortality. Aldosterone receptor antagonists and antigonadotropins did not show any benefit. No significant between-group difference was found in the early or late initiation of therapy. Antiandrogens also reduced hospitalizations and the duration of hospital stay, and improved recovery rates.

Clinckemalie et al., Androgen regulation of the TMPRSS2 gene and the effect of a SNP in an androgen response element. Molecular Endocrinology, Volume 27, Issue 12, pages 2028-2040 (Dec. 1, 2013). TMPRSS2 is positively regulated by androgens.

Diamond et al. Acute Respiratory Distress Syndrome. In: StatPearls (Treasure Island (FL): StatPearls Publishing, January 2023). provides a description of acute respiratory distress syndrome.

Dreicer, Phase 3 VERACITY clinical study of sabizabulin in men with metastatic castrate resistant prostate cancer who have progressed on an androgen receptor targeting agent. Journal of Clinical Oncology, Volume 40, Number 6, suppl. (Feb. 16, 2022). Sabizabulin is an exciting first-in-class agent that will add to the armamentarium for the treatment of metastatic castration and ARTA resistant prostate cancer. A phase 1b/2 clinical study was conducted to establish the MTD and evaluate the preliminary efficacy in men with metastatic castrate resistant prostate cancer (mCRPC) who progressed on at least one androgen receptor targeting agent (ARTA). The most common AEs reported were mild to moderate diarrhea, fatigue, nausea, and vomiting with no clinically relevant neurotoxicity or neutropenia. In the phase 1 b/2, in all evaluable patients that would qualify for the VERACITY study. Median radiographic progression free survival was >12 months in the phase 1b portion including responses >two years. The revised formulation for use in the Phase 3 study, with improved bioavailability, is 32 mg PO qd. VERACITY is an ongoing phase 3 multicenter, randomized, active-control study designed to evaluate sabizabulin in the treatment of mCRPC who progressed on at least one ARTA. Based upon the phase 1b/2 clinical trial, sabizabulin daily chronic oral dosing has a favorable safety profile, is feasible to administer chronically, and has significant and durable antitumor activity. Clinical trial information: NCT04844749.
Evans et al., Surviving Sepsis Campaign: International guidelines for management of sepsis and septic shock 2021. Critical Care Medicine, 49(11), e1063-e143 (2021).
Experton et al., A predictive model for severe COVID-19 in the Medicare population: a tool for prioritizing primary and booster COVID-19 vaccination. Biology, 10(11), 1185 (2021).
Ferguson et al., The Berlin definition of ARDS: An expanded rationale, justification, and supplementary material. Intensive Care Medicine, 8(10), 1573-82 (2012).

Ford et al., Increased androgen binding in keloids: A preliminary communication. J. Dermatol. Surg. Oncol., 9(7), 545-7 (July 1983). Sex hormone binding protein analyses were performed on six keloids, the adjacent skin, and on six simple scars. A high level of androgen binding was found in the keloids. The estrogen and progesterone binding activities were low. Para-keloid tissues have high androgen binding in cytosol protein. Estrogen and progesterone binding activities were also low. In six simple scars, androgen binding was less than 1/10 of that found in the keloids. These data suggests that localized hyperandrogen metabolism may play a causal or at least contributory role in the pathogenesis of keloids.
Fredericks et al., Deep RNA sequencing of intensive care unit patients with COVID-19, Scientific Reports, 12(1) (January 2022).
Goren et al., A preliminary observation: Male pattern hair loss among hospitalized COVID-19 patients in Spain—A potential clue to the role of androgens in COVID-19 severity. J Cosmet Dermatol. 2020 July; 19(7):1545-1547.
Goren et al., J. Eur. Acad. Dermatology Venereol. (2020). When hospitalized due to COVID-19, those on antiandrogens for over 6 months had reduced chances of being admitted to an intensive care unit.
Goren et al., What does androgenetic alopecia have to do with COVID-19? An insight into a potential new therapy. Dermatol. Ther., 33(4), e13365 (July 2020).
Kotani et al., Antiandrogen activity of drugs for COVID-19. The case of sabizabulin. [editorial] Signa Vitae, 19(3), 1-3 (2023).
Krause et al., SARS-CoV-2 variants and vaccines. New England Journal of Medicine, 385, 179-86 (2021).
Lee, Yousaf, Fang, & Kolodney. Male balding is a major risk factor for severe COVID-19. J. Am. Acad. Dermatol. (July 2020).
Letko et al., Functional assessment of cell entry and receptor usage for SARS-CoV-2 and other lineage B betacoronaviruses. Nature Microbiology, Volume 5, pages 562-569 (Feb. 24, 2020) support the idea that TMPRSS2 promotes virus.
Lin et al., Accelerated viral clearance and symptom resolution in symptomatic COVID-19 outpatients treated with antiandrogens.

Manalac, Veru moves forward with late-stage COVID study despite FDA roadblock (Mar. 15, 2023). Veru will conduct a Phase III study of sabizabulin in hospitalized patients with moderate to severe COVID-19 who are at high risk of developing acute respiratory disease syndrome (ARDS). The drug will also enter a Phase III study in hospitalized influenza patients at risk of ARDS. On March 2, the company said the regulator rejected the application but remained open to working with Veru on the drug's development. Concurrent with the rejection, the regulator also provided feedback on Veru's proposed protocol for a confirmatory Phase III study for sabizabulin, stating that the company should provide appropriate time frames for interim analyses.
Mangul et al., ROP: Dumpster diving in RNA-sequencing to find the source of 1 trillion reads across diverse adult human tissues. Genome Biology, 19, 36 (2018).

McCoy et al. Proxalutamide (GT0918) reduces the rate of hospitalization for COVID-19 male outpatients: A randomized double-blinded placebo-controlled trial. Front Med. (2021). The results of a randomized, placebo-controlled trial of proxalutamide 200 mg for up to seven days in a trial showed that the 30-day hospitalization rate was about 2% in men taking proxalutamide compared to about 25% in those taking placebo, a reduction of more than 90% of the risk of hospitalization compared to usual care.
McCoy et al., 5-alpha-reductase inhibitors are associated with reduced frequency of COVID-19 symptoms in males with androgenetic alopecia. J. Eur. Acad. Dermatol Venereol., 35(4), e243-e246 (2021). Among men with diagnosis of AGA, dutasteride users had less symptomatic COVID-19 disease in the outpatient setting.
McCoy et al., Androgen receptor genetic variant predicts COVID-19 disease severity: a prospective longitudinal study of hospitalized COVID-19 male patients. J. Eur. Acad. Dermatol Venereol., 35(1), e15-e17 (2021).
McCoy et al., Racial variations in COVID-19 deaths may be due to androgen receptor genetic variants associated with prostate cancer and androgenetic alopecia. Are anti-androgens a potential treatment for COVID-19?J. Cosmet. Dermatol., 19(7), 1542-1543 (July 2020).
Monaghan et al., Deep RNA sequencing of intensive care unit patients with COVID-19. medRxiv (2021).
Montopoli et al., Androgen-deprivation therapies for prostate cancer and risk of infection by SARS-CoV-2: a population-based study (N=4532). Ann Oncol. 2020 August; 31(8):1040-1045.
NCT04446429 Anti-androgen treatment for COVID-19. Clinicaltrials.gov. Published 2021.
NCT04728802. Supplement: Preliminary Report: Proxalutamide Treatment for Hospitalized COVID-19 Patients (NCT04728802). Report Date: Mar. 9, 2021. Proxalutamide improves lung opacities in hospitalized COVID-19 patients when compared to placebo.
NCT04729491. A double-blinded placebo-controlled trial for antiandrogens in men.

NCT05009732 (Kintor Pharmaceuticals). Kintor Pharmaceuticals, Ltd. manufactures and plans to market proxalutamide, and has an investigational new drug (IND) application under United States Food and Drugs Administration to conduct a Phase 3 study for proxalutamide for COVID-19.

Newton, Veru earns FDA go-ahead for new Phase III Covid-19 trial (May 4, 2023). Veru announced that the US Food and Drug Administration (FDA) provided positive feedback on a newly proposed Phase III trial testing sabizabulin in 408 patients with moderate to severe Covid-19. Veru plans to initiate the trial in the second half of 2023. Previously, the FDA elected not to issue an emergency use authorization for sabizabulin based on a 210-patient Phase III study in Covid-19 (NCT04842747). Though the trial met its primary endpoint of reducing all-cause mortality, regulators had expressed concerns over the strength of the data. In an FDA Advisory Committee meeting on Nov. 9, 2022, a panel of outside experts voted eight-to-five against an emergency use authorisation for sabizabulin. During the meeting, experts questioned the small study size, baseline imbalances between the treatment and placebo groups, and an abnormally high mortality rate in the placebo arm.
Olwal et al., Parallels in sepsis and COVID-19 conditions: Implications for managing severe COVID-19. Frontiers in Immunology, Volume 12, Article 602848 (February 2021).
Patel et al., Does androgen deprivation therapy protect against severe complications from COVID-19? Ann. Oncol., 31:1419-1420 (2020).
Rudd et al., Global, regional, and national sepsis incidence and mortality, 1990-2017: Analysis for the Global Burden of Disease Study. Lancet (London, England), 395(10219), 200-11 (2020).

Satija & Mahobe, U.S.FDA panel votes against Veru's COVID-19 pill. Reuters (Nov. 9, 2022). A panel of outside advisers to the U.S. health regulator on Wednesday voted against authorizing Veru Inc's drug for treating high-risk patients hospitalized with COVID-19, citing multiple concerns over efficacy and safety data being based on a small trial. While data shows sabizabulin can also produce antiviral and anti-inflammatory responses, the U.S. Food and Drug Administration staff reviewers have said its effects against COVID-19 are uncertain. During the meeting, other issues with sabizabulin's data were also discussed, such as the lack of clarity over a relevant patient population and the high rate of deaths in a placebo group, which the reviewers had previously flagged.
Salazar Arenas et al., Alopecia and severity of COVID-19: A cross-sectional study in Peru. Le Infez. Med., 29(1), 37-45 (2021).
Samuel et al., Androgen signaling regulates SARS-CoV-2 receptor levels and is associated with Severe COVID-19 symptoms in men. Cell Stem Cell, 27(6), 876-889.e12 (2020).

Schierle et al., Elevated levels of testosterone receptors in keloid tissue. Plastic and Reconstructive Surgery, 100(2), 390-395 (August 1997). These data suggest that elevated androgen receptor levels exist in clinical active keloid tissue and that possible therapeutic means might include topical antiandrogen therapy.
Shahnoor, Khan, & Habiba, Sabizabulin—an unprecedented yet effective drug against COVID-19. Journal of the Pakistan Medical Association, 73(6), 1363 (2023).
Shimizu et al., Flutamide attenuates pro-inflammatory cytokine production and hepatic injury following trauma-hemorrhage via estrogen receptor-related pathway, Annals of Surgery, Vol. 245, No. 2, pages 297-304 (February 2007). XP055983244.
Stralin et al., Mortality in hospitalized COVID-19 patients was associated with the COVID-19 admission rate during the first year of the pandemic in Sweden. Infect. Dis., 54, 145-151 (2022).
The results of Brazil trial for use in COVID-19 (released on YouTube by Samel Hospital in Brazil, Mar. 10, 2021).
Vazquez-Guillamet et al., Using the number needed to treat to assess appropriate antimicrobial therapy as a determinant of outcome in severe sepsis and septic shock. Critical Care Medicine, 42(11), 2342-9 (2014).

Veru announces FDA grant of fast track designation for sabizabulin for the treatment of hospitalized COVID-19 patients at high risk for acute respiratory distress syndrome. Globe Newswire (Jan. 31, 2022). The Phase 3 clinical trial is a double-blind, multicenter, multinational, randomized (2:1), placebo-controlled trial evaluating daily oral doses of 9 mg sabizabulin for up to 21 days versus placebo in 300 hospitalized patients (200 subjects treated with sabizabulin and 100 subjects receive placebo/standard of care) who have SARS-CoV-2 virus infection and who are at high risk for ARDS. Subjects in the sabizabulin and placebo arms will also be allowed to receive standard of care. The primary efficacy endpoint will be the proportion of patients that die on study up to Day 60. Secondary endpoints will include the proportion of patients without respiratory failure, days in ICU, WHO Ordinal Scale for Clinical Improvement change from baseline, days on mechanical ventilation, days in the hospital, and viral load. Clinical results are expected in the first half of calendar year 2022. In addition, sabizabulin, which has dual anti-viral and anti-inflammatory effects, is currently enrolling in a Phase 3 study for the treatment of hospitalized COVID-19 patients at high risk for acute respiratory distress syndrome, also known as the cytokine storm. Sabizabulin is a cytoskeleton disruptor which blocks microtubule trafficking and has the potential to treat both the SARS-CoV-2 viral infection and the cytokine storm and septic shock that leads to ARDS and the high COVID-19 mortality rates.

Veru reaches agreement with FDA on new phase 3 clinical trial for sabizabulin for broader indication: Hospitalized adult patients with any type of viral acute respiratory distress syndrome (ARDS), Globe Newswire (Sep. 26, 2023). Veru Inc. reached agreement with FDA on single Phase 3 clinical trial design to expand treatment population to include all hospitalized adult patients with any type of virus induced ARDS including the tripledemic viruses: Influenza, RSV, and SARS-CoV-2. Based on high mortality rate for viral ARDS, the expected sabizabulin Phase 3 study size is 408 patients with all-cause mortality at Day 60 as the primary endpoint. The Phase 3 study may suffice as a single study for NDA submission. Veru, Inc. plans to conduct the all comers viral ARDS Phase 3 study instead of the second confirmatory Phase 3 COVID-19 study.

VERU-111 in the Treatment of SARS-Cov-2 Infection by Assessing Its Effect on the Proportion of Patients Who Die on Study. ClinicalTrials.gov (press release) (Apr. 13, 2021).

Veru, Inc. Veru announces date for FDA advisory committee meeting to review emergency use authorization for sabizabulin for hospitalized COVID-19 patients at high risk for acute respiratory distress syndrome. Globe Newswire (Sep. 7, 2022).

Veru Inc., Veru's novel COVID-19 drug candidate reduces deaths by 55% in hospitalized patients in interim analysis of Phase 3 study; Independent data monitoring committee halts study early for overwhelming efficacy. Globe Newswire (Apr. 11, 2022). In a phase III study on the treatment of severe courses of COVID-19, sabizabulin reduced mortality by 55%. The test phase was stopped prematurely so that the drug no longer had to be withheld from the placebo control group. Rabin, New Drug Slashed Deaths Among Patients With Severe Covid, Maker Claims. The New York Times (Apr. 11, 2022). See also Veru Announces Oral Late-Breaking Presentation of Phase 2 Data of Sabizabulin for the Treatment of Hospitalized Severe COVID-19 Patients at High Risk for Acute Respiratory Distress Syndrome at the 32nd European Congress of Clinical Microbiology & Infectious Diseases (press release). Veru Inc., Globe Newswire (Apr. 25, 2022).

Vicenzi et al. The efficacy of the mineralcorticoid receptor antagonist canrenone in COVID-19 Patients. J Clin Med. 2020; 9(9). Canrenone, an active metabolite of spironolactone, was associated with reduced fatalities in an intensive care observational series of cases with hypertension or hypokalemia.
Wambier & Goren, Severe acute respiratory syndrome coronavirus 2 (SARS-CoV-2) infection is likely to be androgen mediated. J. Am. Acad. Dermatol., 83(1), 308-309 (2020).
Wambier et al., Androgen sensitivity gateway to COVID-19 disease severity. Drug Dev Res., 81(7), 771-776 (2020).
Wambier et al., Androgenetic alopecia in COVID-19: Compared to age-matched epidemiologic studies and hospital outcomes with or without the Gabrin sign. J. Am. Acad. Dermatol. (July 2020).
Wambier et al., Androgenetic alopecia present in the majority of patients hospitalized with COVID-19: The Gabrin sign. J. Am, Acad Dermatol., 83(2), 680-682 (August 2020).
Wambier, Mehta, Goren, & Cadegiani, COVID-19, androgens, and androgenic alopecia. Dermatological Rev., der2.50. (2020).

Welén et al., A phase 2 trial of the effect of antiandrogen therapy on COVID-19 outcome. No evidence of benefit, supported by epidemiology and in vitro data. Eur. Urol., 81, 285-93 (2022) provided data from the pilot phase of a trial using enzalutamide for hospitalized COVID-19 patients. Using parallel lines of evidence, the authors argue that all investigations into antiandrogen therapy in COVID-19 should be halted. But this paper actually showed positive effects of enzalutamide for hospitalized COVID-19 patients. See Wambier & Nau, Eur. Urol., 81(6), e141-e142 (June 2022). See also, Welen et al., Reply to Carlos G. Wambier and Gerard J. Nau's Letter to the Editor re: Karin Welen, Ebba Rosendal, Magnus Gisslen, et al. A Phase 2 Trial of the Effect of Antiandrogen Therapy on COVID-19 Outcome: No Evidence of Benefit, Supported by Epidemiology and In Vitro Data. Eur. Urol. 2022, 81:285-93. European Urology, 81, e143-e144 (2022). Positive Effects of Enzalutamide for Hospitalized COVID-19 Patients. First, the principle of analysis of clinical trials is to accept that single observations may be random, and second, the inclusion and exclusion criteria used in the trial make any comparisons or extrapolations to over-all outcomes incorrect. Wambier and Nau further argue that the 5-d treatment duration for enzalutamide contributes to the lack of benefcial effect on the basis of two cases of mechanical ventilation (days 5 and 9). The half-life of enzalutamide is 6 d, meaning that a vanished effect of enzalutamide is unlikely to be the cause of these worse outcomes. In summary, after initial enthusiasm, we now disagree with Wambier and Nau regarding the value of antiandrogens in the treatment of COVID-19. Nevertheless, the results from Brazil are intriguing and we eagerly await results from the HITCH trial investigating the gonadotropin-releasing hormone antagonist degarelix in a similar setting (NCT04397718), which may provide further information regarding the value of androgen inhibition as a treatment for COVID-19.

Whole-genome sequencing as part of national and international surveillance programmes for antimicrobial resistance: A roadmap. BMJ Global Health, 5(11) (2020).
Willyard, How antiviral pill molnupiravir shot ahead in the COVID drug hunt. Nature (2021).
Wu et al., Suppression of androgen receptor (AR)-ACE2/TMPRSS2 axis by AR antagonists may be therapeutically beneficial for male COVID-19 patients. SSRN Electron. J. (April 2020).

Yang et al., Androgen-related disorders and hormone therapy for patients with keloids [Review], Chinese Journal of Plastic and Reconstructive Surgery, Volume 4, Issue 1, March 2022, pages 44-48. Keloids are a fibroproliferative disorder of the skin and can cause physical discomfort and psychological burden. Apart from local factors such as skin tension and infection, increasing evidence suggested that systemic endocrine factors also contribute to the emergence and development of keloids. Hormone disorders have long been suspected to be a risk factor; however, previous studies have mainly focused on the role of female hormones and neglected the critical role of male hormones. Androgens (i.e., male hormones) could become actively involved through sebum-associated hypersensitivity reactions and acne-derived skin lesions, resulting in persistent cutaneous inflammation. This hypothesis was also supported by previous in vitro studies, in which elevated androgen levels and androgenic receptors were detected in keloid tissues. Moreover, relief of pain and pruritus was observed in patients with keloids who accidently received anti-androgen treatment for other irrelevant indications. Androgen-related disorders may be critical in the pathogenesis of keloids, and systemic treatment targeting sex hormones may provide long-term benefits for predisposed patients.
Zong et al., The intersection of COVID-19 and cancer: Signaling pathways and treatment implications, Molecular Cancer, Vol. 20, No. 76, pages 1-19 (May 17, 2021). XP055983245.
Textbooks and technical references: Current Protocols in Immunology (CPI) (2003). John E. Coligan, A D A M Kruisbeek, David H Margulies, Ethan M Shevach, Warren Strobe, (eds.) John Wiley and Sons, Inc. (ISBN 0471142735, 9780471142737).
Current Protocols in Molecular Biology (CPMB), (2014). Frederick M. Ausubel (ed.), John Wiley and Sons (ISBN 047150338X, 9780471503385).
Current Protocols in Protein Science (CPPS), (2005). John E. Coligan (ed.), John Wiley and Sons, Inc.
Immunology (2006). Werner Luttmann, published by Elsevier. Janeway's Immunobiology, (2014). Kenneth Murphy, Allan Mowat, Casey Weaver (eds.), Taylor & Francis Limited, (ISBN 0815345305, 9780815345305).
Laboratory Methods in Enzymology: DNA, (2013). Jon Lorsch (ed.) Elsevier (ISBN 0124199542).
Lewin's Genes XI, (2014). published by Jones & Bartlett Publishers (ISBN-1449659055).
Molecular Biology and Biotechnology: a Comprehensive Desk Reference, (1995). Robert A. Meyers (ed.), published by VCH Publishers, Inc. (ISBN 1-56081-569-8).
Molecular Cloning: A Laboratory Manual, 4th ed., Michael Richard Green and Joseph Sambrook, (2012). Cold Spring Harbor Laboratory Press, Cold Spring Harbor, N.Y., USA (ISBN 1936113414).
Pharmaceutical Sciences 23^{rd} edition (Elsevier, 2020).
The Encyclopedia of Molecular Cell Biology and Molecular Medicine, Robert S. Porter et al., (eds.), published by Blackwell Science Ltd., 1999-2012 (ISBN 9783527600908).

The Merck Manual of Diagnosis and Therapy, 19^{th} edition (Merck Sharp & Dohme Corp., 2018).

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

We claim:

1. A method for treating a condition and/or a sepsis in a subject in need thereof, the method comprising steps of:
(1) executing an analytical test on the subject, whereby the test will assess whether or not the subject is an appropriate candidate for a treatment by an administration of a therapeutically effective amount of an anti-androgen small molecule or a potential anti-androgen small molecule;
(2) if the subject is an appropriate candidate after the test in step (1), administer the therapeutically effect amount of the anti-androgen small molecule and/or the potential anti-androgen small molecule to the subject; and
whereby the condition and/or the sepsis in the subject is improved compared to a ceteris paribus subject without the administering of the therapeutically effect amount of the anti-androgen small molecule and/or the potential anti-androgen small molecule to the subject.

2. The method of claim 1, further comprising the step of: (3) executing an analytical test to assess success of the administration of the anti-androgen small molecule or a potential anti-androgen small molecule, wherein the analytical test comprises an RNA sequencing test, a radiological test, a dexamethasone androgen suppression test, a COVID-19 test, a culture test, a nasopharyngeal viral clearance test, a protein expression test, a biomarker test, a clinical symptom assessment, or a combination thereof.

3. The method of claim 1, wherein the small molecule comprises sabizabulin or a pharmaceutically acceptable hydrate, solvate, and/or salt form of sabizabulin.

4. The method of claim 1, wherein the anti-androgen small molecule or the potential anti-androgen small molecule is administered in a therapeutically effective amount that is determined based on a severity and stage of the condition and/or sepsis in the subject.

5. The method of claim 1, wherein the improvement in the condition and/or sepsis is compared to the ceteris paribus subject without the administering of the therapeutically effective amount of the anti-androgen small molecule or the potential anti-androgen small molecule, and wherein the improvement is measured by one or more clinical parameters or biomarkers.

6. The method of claim 1, wherein the anti-androgen small molecule or the potential anti-androgen small molecule is at least one of an anti-androgen small molecule or a potential anti-androgen small molecule that acts on an androgen receptor pathway.

7. The method of claim 1, wherein the analytical test substantially assesses the appropriateness of the subject for the treatment by measuring one or more biomarkers, clinical symptoms, or risk factors associated with the condition and/or sepsis.

8. The method of claim 1, wherein the administering is substantially of the therapeutically effective amount of the anti-androgen small molecule or the potential anti-androgen small molecule, and wherein the therapeutically effective amount is sufficient to improve the condition and/or sepsis in the subject.

9. The method of claim 1, wherein the condition comprises COVID-19 infection, a traumatic lung injury, a skin condition, or any sign, symptom or condition that mimics a sepsis infection.

10. The method of claim 1, wherein the condition and/or sepsis is one or more of a bacterial infection, viral infection, fungal infection, parasitic infection, inflammatory condition, or sepsis.

11. The method of claim 1, wherein the subject is a human patient diagnosed with or at risk of developing the condition and/or sepsis.

12. The method of claim 1, wherein the analytical test is performed prior to the administering and is used to select subjects who are likely to respond to the treatment.

13. The method of claim 1, wherein the administering is performed after the assessing and only in subjects who are determined to be appropriate candidates for the treatment based on one or more results of the analytical test.

14. The method of claim 1, wherein the improvement is assessed after the administering at one or more time points to monitor the effectiveness of the treatment.

15. The method of claim 1, wherein the anti-androgen small molecule or the potential anti-androgen small molecule is administered orally, intravenously, intramuscularly, or subcutaneously using a suitable pharmaceutical formulation.

16. The method of claim 1, wherein the anti-androgen small molecule or the potential anti-androgen small molecule is administered in a single dose or in multiple doses according to a therapeutic regimen that is tailored to an individual subject.

17. The method of claim 1, wherein the therapeutically effective amount is determined based on a severity of the condition and/or sepsis in the subject, as well as other clinical factors such as age, weight, and overall health of the subject.

18. The method of claim 1, wherein the anti-androgen small molecule or the potential anti-androgen small molecule is selected based on one or more results of the analytical test, and wherein different anti-androgen small molecules or potential anti-androgen small molecules may be selected for different subjects depending on their individual test results.

19. The method of claim 1, wherein the analytical test assesses whether or not the subject is an appropriate candidate for the treatment by evaluating one or more genetic, biochemical, or clinical parameters that are predictive of a subject's response to the treatment.

20. The method of claim 1, wherein the therapeutically effective amount is administered to the subject assessed as an appropriate candidate for the treatment, and wherein the subject is monitored for adverse reactions or side effects during a course of the treatment.
